(19) United States
(12) Patent Application Publication
(10) Pub. No.: US 2025/0037848 A1
(43) Pub. Date: Jan. 30, 2025

(54) SYSTEMS, METHODS, AND MEDIA FOR DYNAMIC SCHEDULING FOR MOBILE SERVICE PROVIDER

(71) Applicant: KARE MOBILE, INC., Louisville, KY (US)

(72) Inventor: Kwane Watson, Louisville, KY (US)

(21) Appl. No.: 18/709,260

(22) PCT Filed: Nov. 10, 2022
(86) PCT No.: PCT/US2022/049588

§ 371 Date: May 10, 2024

Related U.S. Application Data
(60) Provisional application No. 63/277,915, filed on Nov. 10, 2021.

(51) Int. Cl. G16H 40/20; G06Q 10/0631; G06Q 40/08

(52) U.S. Cl. CPC G16H 40/20; G06Q 10/06311; G06Q 40/08

(57) ABSTRACT

In accordance with some embodiments, systems, methods, and media for using positional information for a mobile vehicle to generate dynamic scheduling are provided. The systems, methods, and media includes: receiving a scheduling request for a procedure of a patient; dynamically receiving the positional information from the mobile vehicle; dynamically determining a production per drive score for a time slot of a plurality of time slots for the procedure based on a production yield for the procedure, an estimated travel time to the procedure, a procedure time for the procedure, and the positional information; dynamically determining a availability level indication of the time slot for the procedure based on the production per drive score of the time slot for the procedure and the scheduling information; dynamically displaying the availability level indication on a graphical user interface; and dynamically transmitting, to the mobile vehicle, procedure information comprising the time slot.

### CROSS-REFERENCE TO RELATED APPLICATIONS

This application claims the benefit of U.S. Provisional Patent Application Ser. No. 63/277,915, filed Nov. 10, 2021, the disclosure of which is hereby incorporated by reference in its entirety, including all figures, tables, and drawings.

### BACKGROUND

Current scheduling systems are only for procedures in a building or a fixed location. In addition, the current scheduling systems can schedule procedures a few weeks from the current time. Thus, patients would not access procedures in near future and/or in a place at or near patients' preferred locations. What are needed are systems and methods for dynamic procedure scheduling for mobile service providers.

### SUMMARY

In accordance with some embodiments of the disclosed subject matter, systems, methods, and media for using positional information for a mobile vehicle to generate dynamic scheduling are provided. The systems, method, and media include steps of: receiving a scheduling request for a procedure of a patient, the procedure configured to occur inside of, in proximity to, or in connection with the mobile vehicle; obtaining scheduling information for the mobile vehicle corresponding to a preferred provider; dynamically receiving the positional information from the mobile vehicle, the positional information comprising a current location of the mobile vehicle; dynamically determining a first production per drive score for a first time slot of a plurality of time slots for the procedure based on a production yield for the procedure, a first estimated travel time to the procedure, a procedure time for the procedure, and the positional information; dynamically determining a first availability level indication of the first time slot for the procedure based on the first production per drive score of the first time slot for the procedure and the scheduling information; dynamically displaying the first availability level indication on a graphical user interface; dynamically transmitting, to the mobile vehicle, procedure information comprising the first time slot; and displaying the procedure information on the graphical user interface.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a system for scheduling procedures in accordance with some embodiments of the disclosed subject matter.

FIG. 2 shows an example of hardware that can be used to implement a computing device and a server, shown in FIG. 1 in accordance with some embodiments of the disclosed subject matter.

FIG. 3 illustrates an example of features that may be available to a consumer using a scheduler according to some embodiments of the present disclosure.

FIG. 4 illustrates another example of features that may be available to a consumer using a scheduler according to some embodiments of the present disclosure.

FIG. 5 illustrates an example architecture for a scheduling system according to some embodiments of the present disclosure.

FIG. 6 illustrates an example login screen for a web-based application according to some embodiments of the present disclosure.

FIG. 7 illustrates a flowchart illustrating an example method for assigning tiered ratings according to some embodiments of the present disclosure.

FIG. 8 illustrates a Red or lowest Tier recommendation for a procedure displayed on a screen, as generated by an example embodiment of the present disclosure.

FIG. 9 illustrates an Orange or intermediate Tier recommendation for a procedure displayed on a screen, as generated by an example embodiment of the present disclosure.

FIG. 10 illustrates a Green or highest Tier recommendation for a procedure displayed on a screen, as generated by an example embodiment of the present disclosure.

FIG. 11 illustrates a home screen dashboard according to some embodiments of the present disclosure.

FIG. 12 illustrates a patient tab, such as the patient tab from the home screen dashboard of FIG. 11.

FIG. 13 illustrates a schedule tab with a list view, such as the schedule tab from the home screen dashboard of FIG. 11.

FIG. 14 illustrates a schedule tab with a calendar view, such as the schedule tab from the home screen dashboard of FIG. 11.

FIG. 15 illustrates an edit/rescheduling-procedure view according to some embodiments of the present disclosure.

FIG. 16 illustrates a cancel-procedure view according to some embodiments of the present disclosure.

FIG. 17 illustrates a providers tab, such as the providers tab from the home screen dashboard of FIG. 11.

FIG. 18 illustrates an add-provider view according to some embodiments of the present disclosure.

FIG. 19 illustrates a user-list view according to some embodiments of the present disclosure.

FIG. 20 illustrates an add-user dashboard according to some embodiments of the present disclosure.

FIG. 21 illustrates a procedure-list view generated in accordance with some embodiments of the present disclosure.

FIG. 22 illustrates a serviceable-areas view generated in accordance with some embodiments of the present disclosure.

FIG. 23 illustrates a procedure-checklist view generated in accordance with some embodiments of the present disclosure.

FIG. 24 illustrates a queue-list view generated in accordance with some embodiments of the present disclosure.

FIG. 25 illustrates a home screen for a mobile application according to some embodiments of the present disclosure.

FIG. 26 illustrates a procedure-detail view for a mobile application in accordance with some embodiments of the present disclosure.

FIG. 27 illustrates a procedure-list view for a mobile application according to some embodiments of the present disclosure.

FIG. 28 illustrates a calendar view for a mobile application according to some embodiments of the present disclosure.

FIG. 29 illustrates a notifications view for a mobile application according to some embodiments of the present disclosure.

FIG. 30 illustrates a profile view for a mobile application according to some embodiments of the present disclosure.

FIG. 31 illustrates a change-password view for a mobile application according to some embodiments of the present disclosure.

FIG. 32 illustrates a flow chart of a mobile application according to some embodiments of the present disclosure.

FIG. 33 illustrates a flow chart of a sign-in process according to some embodiments of the present disclosure.

FIG. 34 illustrates a flow chart of a forgot-password process according to some embodiments of the present disclosure.

FIG. 35 illustrates a flow chart for scheduling a procedure according to some embodiments of the present disclosure.

FIG. 36 illustrates a flow chart for a procedure scheduling process according to some embodiments of the present disclosure.

FIG. 37 illustrates a flow chart for a mobile application architecture according to some embodiments of the present disclosure.

FIG. 38 illustrates a data relationship of a scheduling database according to some embodiments of the present disclosure.

FIG. 39 illustrates a flow chart for using positional information for a mobile vehicle to generate dynamic scheduling according to some embodiments of the present disclosure.

FIG. 40 illustrates an example scenario based on a scheduling algorithm according to some embodiments of the present disclosure.

FIG. 41 illustrates another example scenario based on a scheduling algorithm according to some embodiments of the present disclosure.

### DETAILED DESCRIPTION

In accordance with various embodiments, mechanisms (which can, for example, include systems, methods, and media) for scheduling procedures for a mobile service provider are provided. In some embodiments, mechanisms described herein can be used to schedule procedures at a time that is optimized for a service provider and a client's respective schedules, as well as taking into account the relative locations of the service provider and client and travel time to commute the distance therebetween. In some examples, the disclosure may be applicable to medical or dental goods or a provider of medical or dental services in connection with a mobile vehicular platform. However, it should be appreciated that the disclosure may be used for any type of goods or service provider that operates in connection with a mobile vehicular platform.

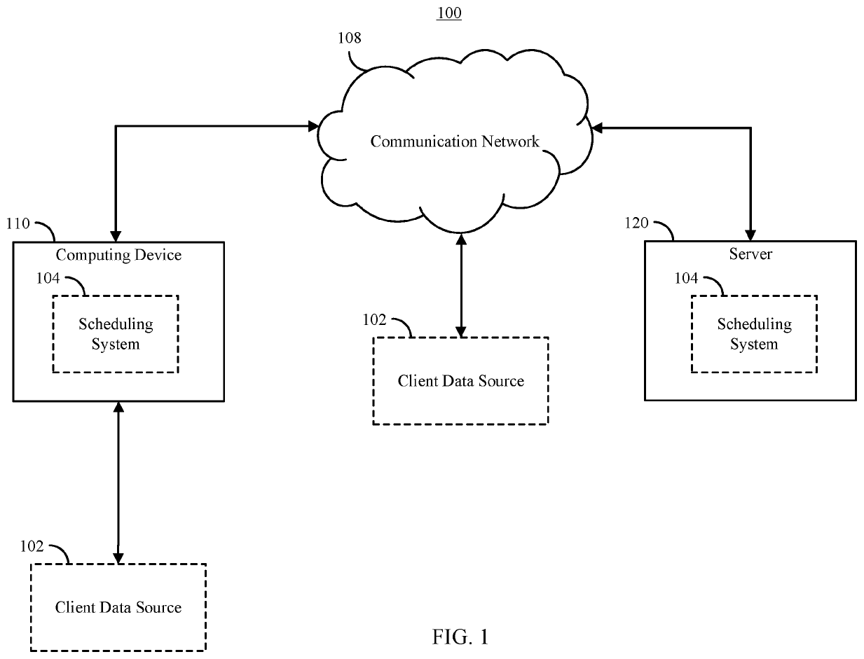

FIG. 1 shows an example 100 of a system for scheduling procedures in accordance with some embodiments of the disclosed subject matter. As shown in FIG. 1, a computing device 110 can receive client data from a client data source 102. In some embodiments, computing device 110 can execute at least a portion of a scheduling system 104 to schedule a procedure for a client. In some embodiments, computing device 110 can execute at least a portion of the scheduling system 104 to analyze client data and schedule procedures at optimized times using mechanisms described herein.

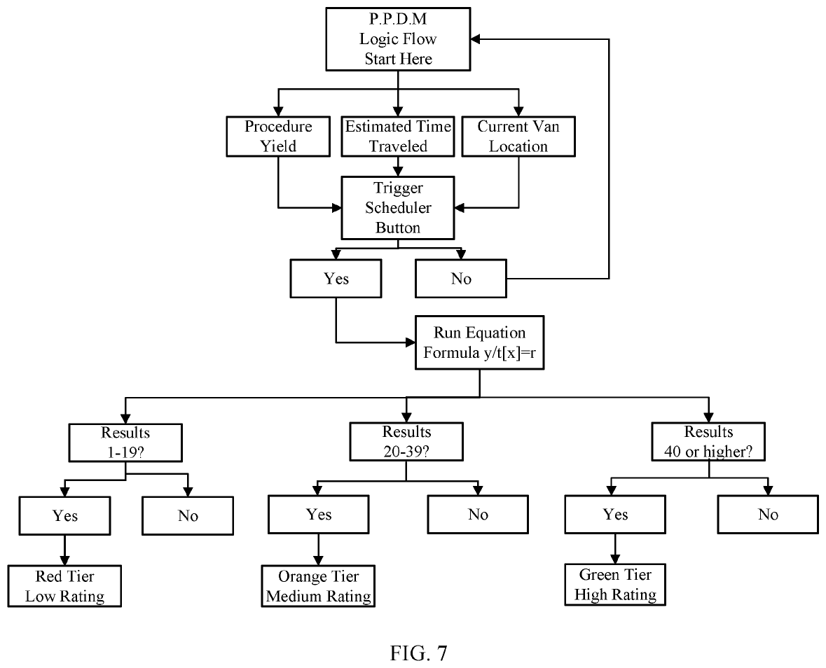

Additionally or alternatively, in some embodiments, computing device 110 can communicate data received from client data source 102 to a server 120 over a communication network 108, which can execute at least a portion of scheduling system 104. In such embodiments, server 120 can return information to computing device 110 (and/or any other suitable computing device) indicative of an optimized schedule time for a client on a given service provider's schedule. In some embodiments, scheduling system 104 can execute one or more portions of the steps shown in FIGS. 7 and 32-36, and described below.

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, etc.

In some embodiments, client data source 102 can be any suitable source of client data (e.g., data provided by a user profile, data provided directly from a user, data provided by a secretary, etc.). In a more particular example, client data source 102 can include memory storing client data (e.g., local memory of computing device 110, local memory of server 120, cloud storage, portable memory connected to computing device 110, portable memory connected to server 120, etc.). In another more particular example, client data source 102 can include an application configured to generate client data (e.g., a scheduling application being executed by computing device 110, server 120, and/or any other suitable computing device).

In some embodiments, client data source 102 can be local to computing device 110. For example, client data source 102 can be incorporated with computing device 110 (e.g., computing device 110 can include memory that stores client data, and/or can execute a program that collects or generates client data). As another example, client data source 102 can be connected to computing device 110 by a cable, a direct wireless link, etc. Additionally or alternatively, in some embodiments, client data source 102 can be located locally and/or remotely from computing device 110, and can communicate client data to computing device 110 (and/or server 120) via a communication network (e.g., communication network 108).

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 108 can be a local area network (LAN), a wide area network (WAN), a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

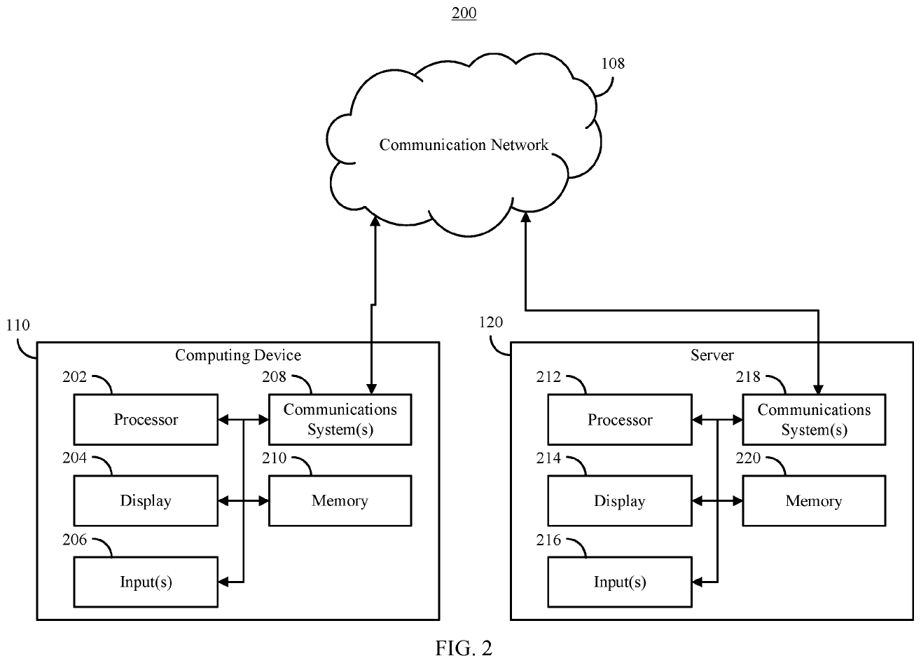

FIG. 2 shows an example 200 of hardware that can be used to implement computing device 110 and/or server 120 in accordance with some embodiments of the disclosed subject matter. As shown in FIG. 2, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), an application specific integrated circuit (ASIC), a field-programmable gate array (FPGA), etc. In some embodiments, display 204 can include any suitable display devices, such as a mobile device screen, computer monitor, a touchscreen, a television, etc. In some embodiments, inputs 206 can include any suitable input devices and/or sensors that can be used to receive user input, such as a keyboard, a mouse, a touchscreen, a microphone, etc.

In some embodiments, communications systems 208 can include any suitable hardware, firmware, and/or software for communicating information over communication network 108 and/or any other suitable communication networks. For example, communications systems 208 can include one or more transceivers, one or more communication chips and/or chip sets, etc. In a more particular example, communications systems 208 can include hardware, firmware and/or software that can be used to establish a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to perform a computer vision task, to present content using display 204, to communicate with server 120 via communications system(s) 208, etc. Memory 210 can include any suitable volatile memory, non-volatile memory, storage, or any suitable combination thereof. For example, memory 210 can include random access memory (RAM), read-only memory (ROM), electronically-erasable programmable read-only memory (EEPROM), one or more flash drives, one or more hard disks, one or more solid state drives, one or more optical drives, etc. In some embodiments, memory 210 can have encoded thereon a computer program for controlling operation of computing device 110. For example, in such embodiments, processor 202 can execute at least a portion of the computer program to transmit client data to server 120, receive client data from server 120, transmit location data to server 120, receive mapping data from server 120, schedule procedures onto a schedule, output procedures onto a display, etc. As another example, processor 202 can execute at least a portion of the computer program to implement scheduling system 104. As yet another example, processor 202 can execute at least a portion of the steps shown in FIGS. 7 and 32-36, and described below.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an ASIC, an FPGA, etc. In some embodiments, display 214 can include any suitable display devices, such as a mobile device screen, a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 108 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, etc.

In some embodiments, memory 220 can include any suitable storage device or devices that can be used to store instructions, values, etc., that can be used, for example, by processor 212 to present content using display 214, to communicate with one or more computing devices 110, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. For example, in such embodiments, processor 212 can receive client data from computing device 110, transmit client data to computing device 110, determine an optimal procedure time for a client on a schedule, transmit results related to the optimal procedure time to computing device 110, and/or cause results related to the schedule or procedure time to be presented (e.g., by computing device 110). As another example, processor 212 can execute at least a portion of the computer program to implement scheduling system 104. As yet another example, processor 212 can execute at least a portion of the steps shown in FIGS. 7 and 32-36, and described below.

Embodiments of the present disclosure may generally be helpful to easily plan procedures in a website, interface, or user portal (e.g., a web dashboard, a mobile application, a computer application, etc.). One mechanism for scheduling procedures described herein may include a calculation algorithm entitled “P.P.D.M” (Production Per Drive Minute), which may assist administrative users to determine whether a recommended procedure is scheduled for an optimal use case (e.g., by increasing provider productivity and travel efficiency for providers and/or consumers to prevent rescheduling and or rerouting). Mechanisms described herein may include the ability to contact patients via phone, and the ability to obtain directions from one procedure to the next via mapping information received from a server. For example, the mapping information may be received from a mapping or routing API that takes as input geographic location information from a user and provides routing information to one or more destinations.

Some embodiments of the present disclosure may include a client application and an admin application. A client may be a mobile computing device (e.g., a smartphone). Alternatively, the client may be another type of computing device as described above with respect to computing device 110. The admin application may be accessible via the Internet (e.g., a website, a virtual machine, server access, etc.). Alternatively, the admin application may be local to a computing device as described above in connection with scheduling system 104. The admin application may receive all relevant client information (e.g., the information of client data source 102). Further, the admin application may receive all relevant scheduler information.

Some embodiments of the present disclosure may include a super administrator, an admin, and a user. The super administrator may have access to the front-end and backend of an application. The super administrator may have access to Super Admin metadata, Admin metadata, User metadata, and application codebase. The super administrator may also have access to features such as the “Providers”, “Procedure Checklist”, “Procedures List”, “Provider Serviceable Areas”, and “Passwords” features.

Some embodiments of the present disclosure may include an admin or administrator. The administrator may have access to the front-end and backend of an application. The administrator may have access to Admin metadata. The administrator may also have access to features such as the “Providers”, “Procedure Checklist”, “Procedures List”, “Provider Serviceable Areas” and “Passwords” features. Some embodiments of the present disclosure may include a user. Users may have access to the front-end of an application. User may further have access to user metadata.

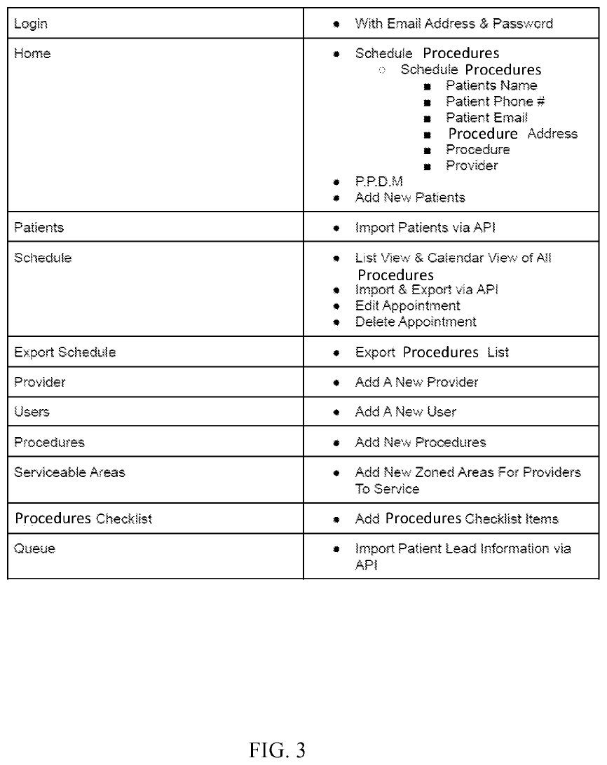

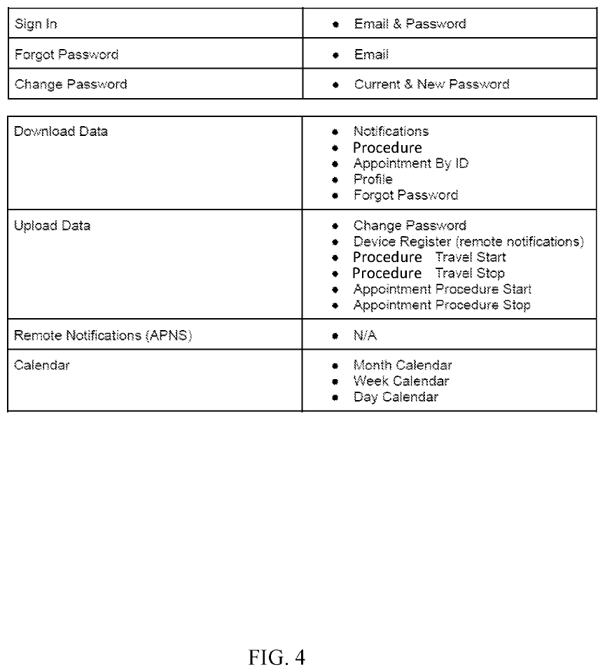

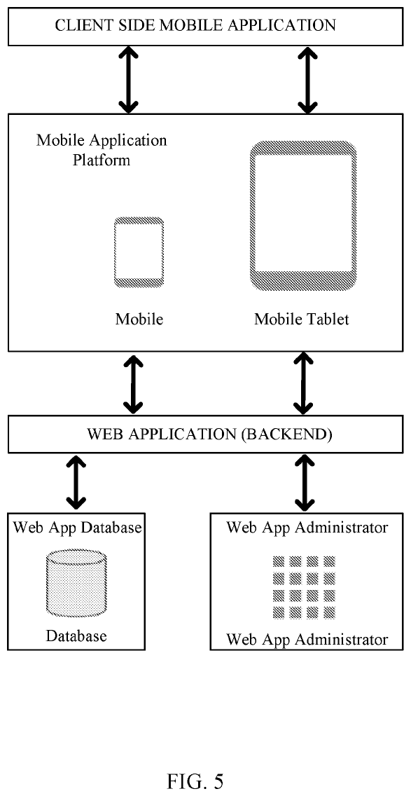

FIG. 3 illustrates an example of features that may be available to a consumer using a scheduler according to some embodiments of the present disclosure. For example, the features illustrated in FIG. 3 may be used in a web-based application. FIG. 4 illustrates another example of features that may be available to a consumer using a scheduler according to some embodiments of the present disclosure. For example, the features illustrated in FIG. 4 may be used in a mobile application. FIG. 5 illustrates an example architecture for a scheduling system according to some embodiments of the present disclosure.

Embodiments of the present disclosure may be used in multiple locations. In an example embodiment of the present disclosure, the scheduling system 104 may be accessed via a personal computer in a residential internet, via an internet website. Both the client and administrator applications discussed earlier herein may be available to consumers anywhere there is an internet connection. Alternatively, the client and administrator applications discussed earlier herein may be available to consumers without an internet connection, and instead, via a wired connection.

A web application in accordance with some embodiments of the present disclosure may be accessible via a uniform resource locator (URL) and may provide functionality to users. Users may be required to obtain or input an authorization key to utilize the functionality of the web application. Users for the web application may enter or login through a system authorization portal. A user account generated may be created and validated by a Super Administrator within the system. Initial user account activation may be triggered using one or more methods. According to some embodiments disclosed herein, user account activation may be triggered by a Super Administrator override. Alternatively, or additionally, user account activation may be triggered by email activation by an end user.

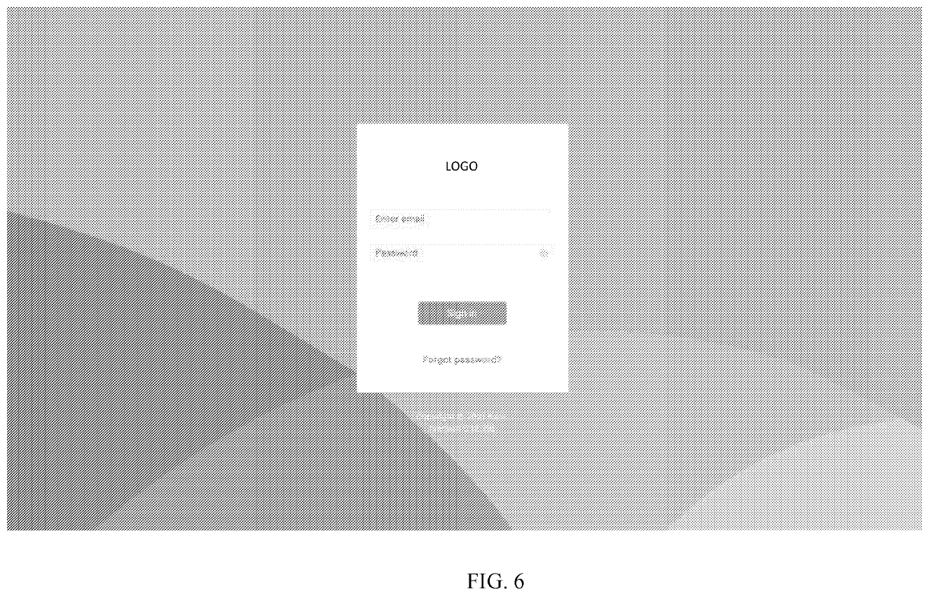

FIG. 6 illustrates an example login screen for a web-based application according to some embodiments of the present disclosure. The login screen may provide users with the option to enter their email, enter their password, sign in, or notify the web-based application that the user has forgotten their password. Upon logging in and verifying the user's credentials, the system may generate and display a home screen to the user. In order for a user to schedule a procedure using mechanisms disclosed herein, some embodiments of the present disclosure may receive several parameters (e.g., via user input at the home screen) to trigger the P.P.D.M. algorithm (discussed earlier herein). The parameters may include: patient first name, patient last name, patient phone number, patient email (if applicable), procedure address, provider, and procedure. Once a user has entered all necessary parameters of information, the user may choose a desired date to schedule a procedure. When a date is chosen, embodiments of the present disclosure may color code all pre-existing procedures to a monochromatic palate of grey on a calendar displayed on a screen. The color coding of the pre-existing procedures may quickly visually notify the user of a scheduling recommendation generated by mechanisms disclosed herein.

A user may click on a “run schedule” button or first button in order to trigger mechanisms disclosed herein for assisting in selecting an optimized procedure on a schedule. Once the first button is clicked, embodiments of the present disclosure may locate all available time slots categorized within the P.P.D.M. model. Each time slot that is available may be color coded one of three colors (e.g., Red, Orange, or Green) in accordance with a respective P.P.D.M. rating, although it will be appreciated that a greater or smaller number of categorizations may be available and that the categorizations may be distinguished from each other in a manner other than by color.

A user may be able to choose a desired time slot from a recommended choice provided by embodiments of the present disclosure. Recommended choices may include available dates and times within a span from a current date generated, to fifteen days in the future. Once a procedure date and time are chosen, the user may confirm the procedure by clicking on the “confirm” button or second button. The “confirm” button may be located on a bottom right corner of a display. According to some embodiments of the present disclosure that use a mobile device, once a procedure has been confirmed, users may be immediately notified of a current status of the procedure (e.g., via a calendar notification, or other display output showing information about the procedure).

FIG. 7 illustrates a flowchart illustrating an example method for assigning tiered ratings according to some embodiments of the present disclosure. As discussed earlier herein, P.P.D.M is an acronym for “Production Per Drive Minute”. Embodiments of the present disclosure may calculate at least the following three parameters: current vehicle location, procedure yield, and estimated travel time. (In some instances, the vehicle may be a van or other vehicle, and it will be understood that the two terms should be used interchangeably herein to describe a mobile vehicle. The vehicle may be configured not just to transport the service provider from one location to the next but also to have a working space in which the service provider can render the requested services. In other instances, the service provider may provide the requested services outside the vehicle, although the vehicle may include the equipment necessary for the service provider to provide the requested services. In still other instances, at least one piece of equipment needed to provide the requested services may not be included in the vehicle, and it may be necessary for the vehicle to travel to an intermediate destination to obtain that equipment prior to traveling to the procedure location.) The three parameters may result in a three tiered color coded rating system according to Equation 1 below. Equation 1 can be used to calculate a P.P.D.M. rating or rating.

$$Procedure Yield / Estimated Travel Time + [ Current Van Location ] = P . P . D . M . Rating \qquad ( 1 )$$

The current van location parameter may be input by a provider (e.g., an address of a current van location, or geographic coordinates of a current van location). Alternatively, or additionally, the current van location may be obtained using features of one or more computing devices. For example, the current van location may be received from a sensor on one or more computing devices. The sensor may be a global positioning system (GPS), a satellite transponder from which geographic position can be calculated using one or more satellite, or a cellular transponder from which geographic position can be calculated using one or more cellular towers. In accordance with some embodiments of the present disclosure, a computing device that has a processor configured to execute computer programmable instructions using mechanisms described herein may receive a current van location from a GPS of the computing device. The computing device may store, via the processor, the current van location (e.g., in volatile, or non-volatile memory of the computing device). The van location may be input into Equation 1 (e.g., by the processor) to calculate the P.P.D.M Rating.

The estimated travel time parameter may be input by a user or provider (e.g., an estimation of travel time from the user's position to the provider's position based on a user or provider's commuting knowledge in a geographic area). Alternatively, or additionally, the estimated travel time may be obtained using features of one or more computing devices. For example, a processor on one or more of the one or more computing devices may receive a location of the user (e.g., a street address, or geographic coordinates of the user) and a location of the provider (e.g., a street address, or geographic coordinates of the provider). The processor may then execute computer programmable instructions to calculate an estimated travel time between the user and the provider (e.g., by using Dijkstra's algorithm and publicly available knowledge regarding road lengths and speed limits, or using another route planning algorithm available to those of ordinary skill in the art).

Alternatively, or additional, the processor may send the location of the user and the location of the provider to a remote computing device (e.g., a server, a virtual machine, or a remote computer). The remote computing device may include an application programming interface (API) that allows the processor to receive information from the remote computing device via classes or libraries of a programming interface. The processor may be required to provide an API key or authentication key to the remote computing device to interface with classes and libraries of the API, and therefore receive information from the API. The processor may receive from the remote computing device routing information including: a fastest route between the user and the provider, distance of the fastest route, travel time of the fastest route, a shortest (e.g., fewest miles) route between the user and the provider, distance of the shortest route, travel time of the shortest route, and traffic information (e.g., delays, or street closures) regarding specific routes. The estimated travel time may be the travel time of the fastest route. Further, the estimated travel time may be adjusted based on the traffic information. For example, in some embodiments of the present disclosure, a user may use a smartphone to schedule a procedure using mechanisms described herein, and a processor on the smartphone may provide the user's location (e.g., the user's current location, or an address, such as a home address of the user) and the provider's location (e.g., the provider's current location, or an address, such as a work address of the provider, or the van location) based on a routing API on a remote server. The smartphone may then receive from the maps API an estimated travel time between the user and the provider. The estimated travel time may be stored (e.g., in volatile, or non-volatile memory of the smartphone, or another computing device). Further, the estimated travel time may be input into Equation 1 (e.g., by the processor) to calculate the P.P.D.M Rating.

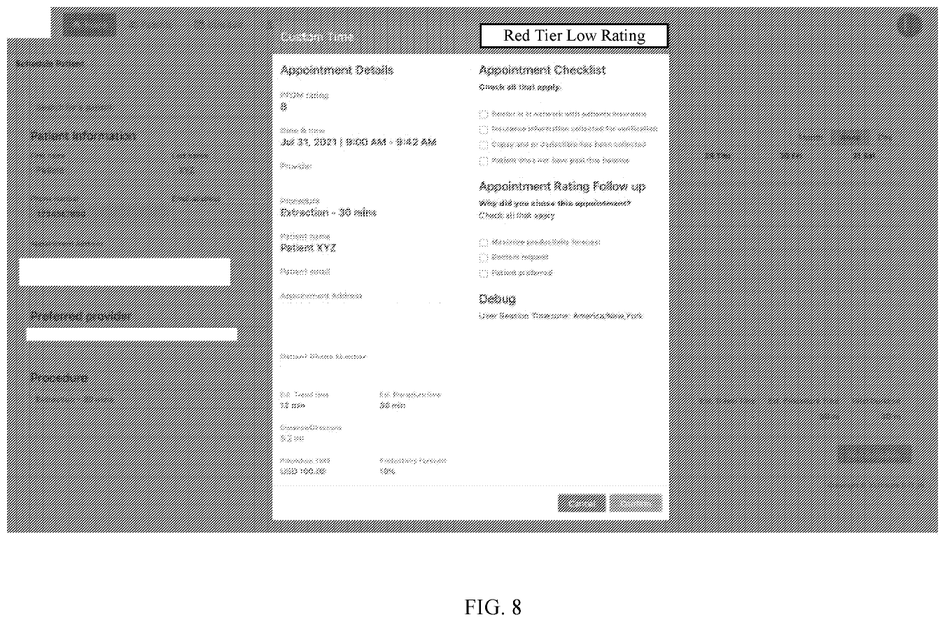

FIG. 8 illustrates a Red or lowest Tier recommendation for a procedure displayed on a screen, as generated by an example embodiment of the present disclosure. The Red Tier recommendation may occur if the P.P.D.M. rating is between 1 and 19. Red Tier recommendations may be the result of low provider production procedure yields that require for the provider to engage in higher than average travel times to patients. Red Tier recommendations are the least efficient (e.g., least optimized) method for users/providers to consider when scheduling procedures. Red Tier recommendations for procedures may be chosen by an end user, if the end user desires; however, Red Tier procedures may be not recommended.

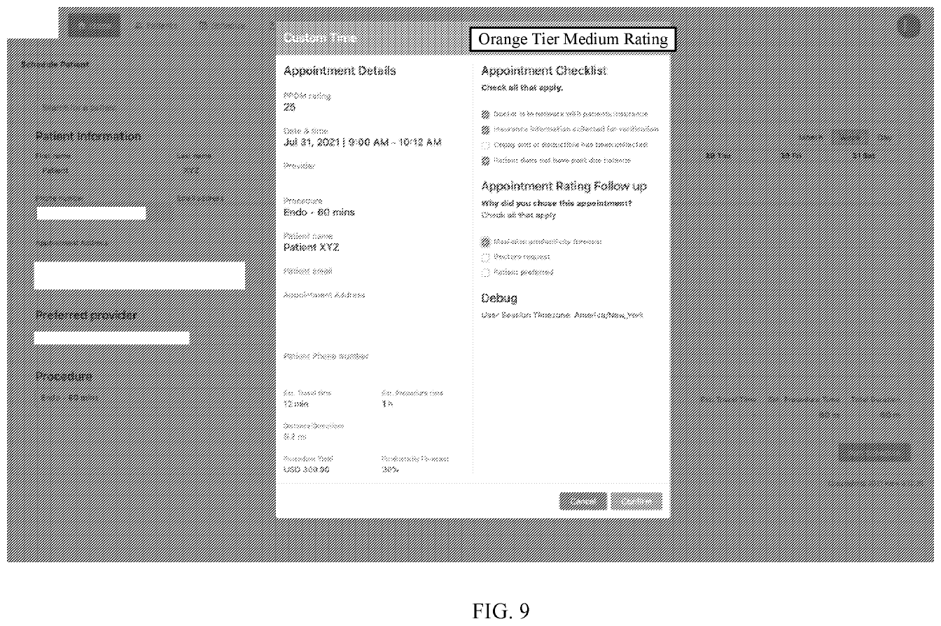

FIG. 9 illustrates an Orange or intermediate Tier recommendation for a procedure displayed on a screen, as generated by an example embodiment of the present disclosure. The Orange Tier recommendation may occur if the P.P.D.M. rating is between 20 and 39. Orange Tier recommendations may be the result of medium provider production procedure yields that require for the provider to engage in average and normal travel time to the patient (e.g., as may be common for users/providers to consider when scheduling procedures). Orange Tier recommendations for procedures may be chosen by an end user, if the end user desires. In some embodiments, additional colors may represent additional intermediate tiers, which may be based on different ratings or rating scales.

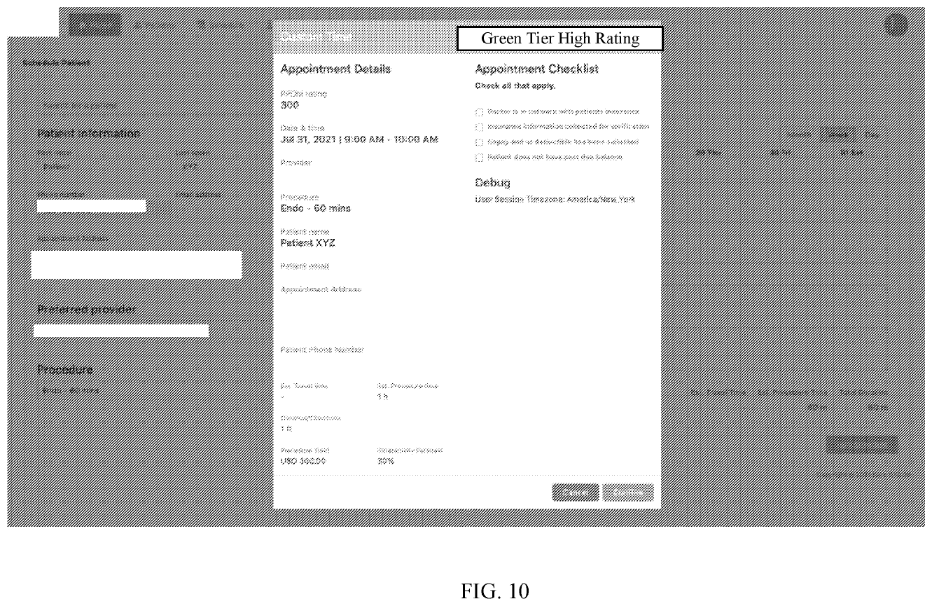

FIG. 10 illustrates a Green or highest Tier recommendation for a procedure displayed on a screen, as generated by an example embodiment of the present disclosure. The Green Tier recommendation may occur if the P.P.D.M. rating is greater than 40. Green Tier recommendations may be the result of high provider production procedure yields that require for the user/provider to engage in the least amount of travel time to the patient. Green Tier recommendations are the most optimal and most desired method for providers to consider when scheduled procedures. Green Tier recommendations for procedures may be chosen by an end user, if the end user desires.

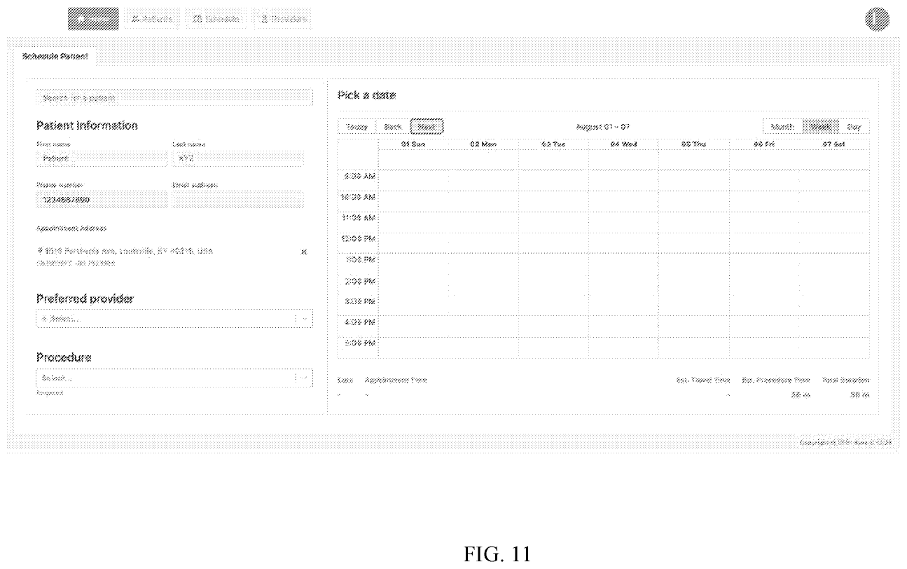

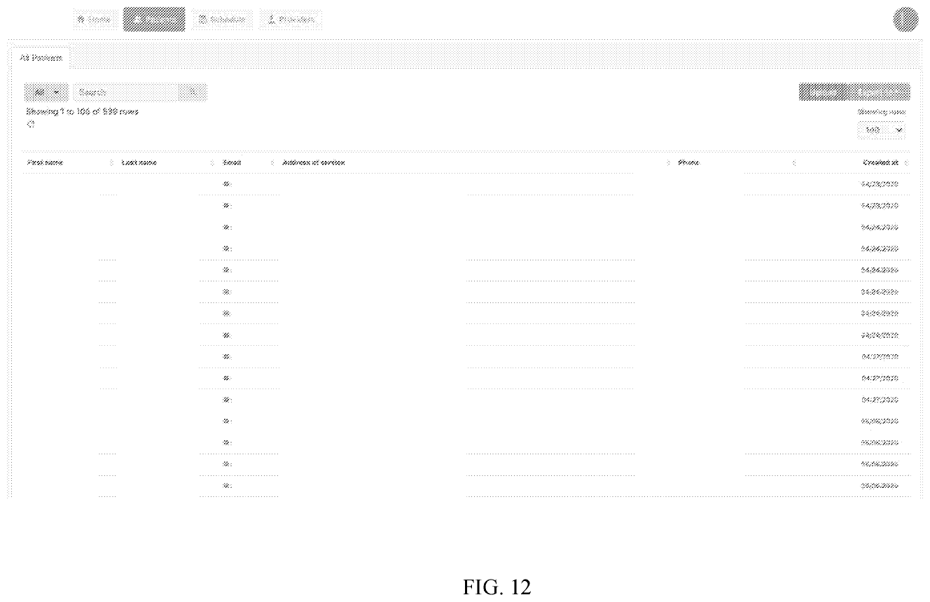

FIG. 11 illustrates a home screen dashboard according to some embodiments of the present disclosure. The home screen dashboard may include a patient tab. The home screen dashboard may further include a schedule tab. The home screen dashboard may further include a providers tab. Generally, a user may navigate between the tabs by selecting one of the respective tabs (e.g., using a touchscreen, via keyboard elements, via a mouse on a computer screen, or via another form of input to a computing device). FIG. 12 illustrates a patient tab, such as the patient tab from the home screen dashboard of FIG. 11. The patient tab may serve as an active database of patient contact information. The following data field can be searched for and sorted (with exception to the “created at” field): First Name, Last Name, Address with Zip Code, Phone Number, Email, Created At.

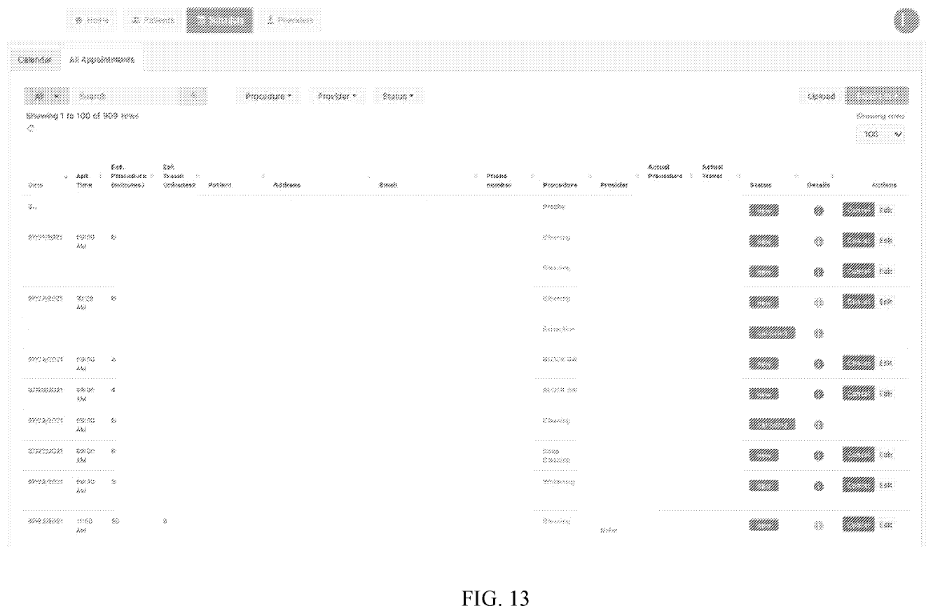

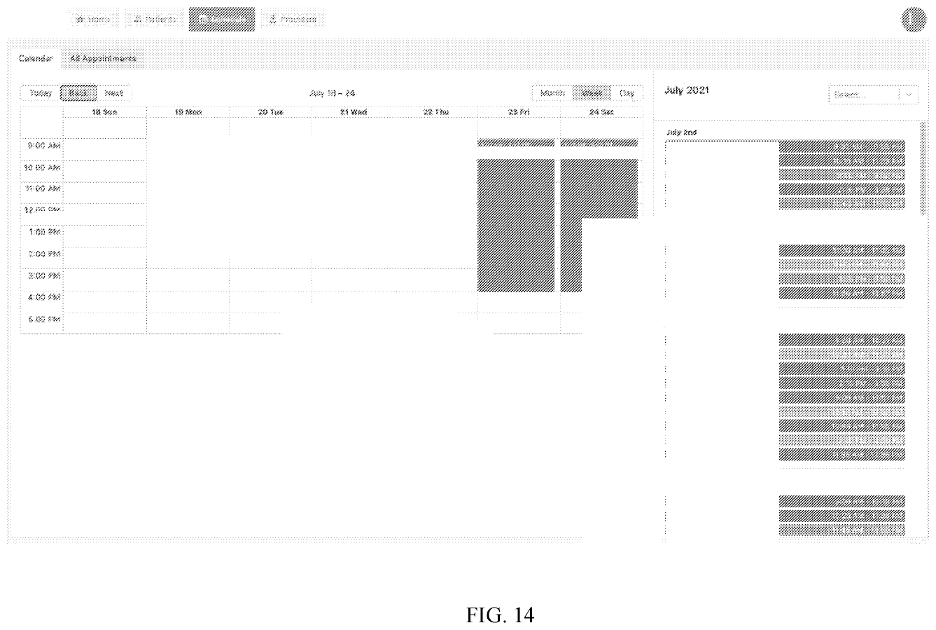

FIGS. 13 and 14 illustrates a schedule tab, such as the schedule tab from the home screen dashboard of FIG. 11. The schedule tab may provide a user with a list view of the practice (as shown in FIG. 13). Additionally, or alternatively, in some embodiments of the present disclosure, the schedule tab may provide a user with a calendar view of the practice (as shown in FIG. 14). The list view may enable users to search, view, sort, and filter through procedures that have been scheduled. The calendar view may enable users to view, sort, and filter through procedures according to a chosen provider. Schedule list view filter options may include: Procedure, Provider, or Status. The Status filter option may further include: New, Travel, Procedure, Completed, and Canceled. Schedule calendar view filter options may include: Provider. Schedules lists may also be downloaded by a user or a system to obtain client data. For example, the schedule list may be downloaded in comma-separated value format.

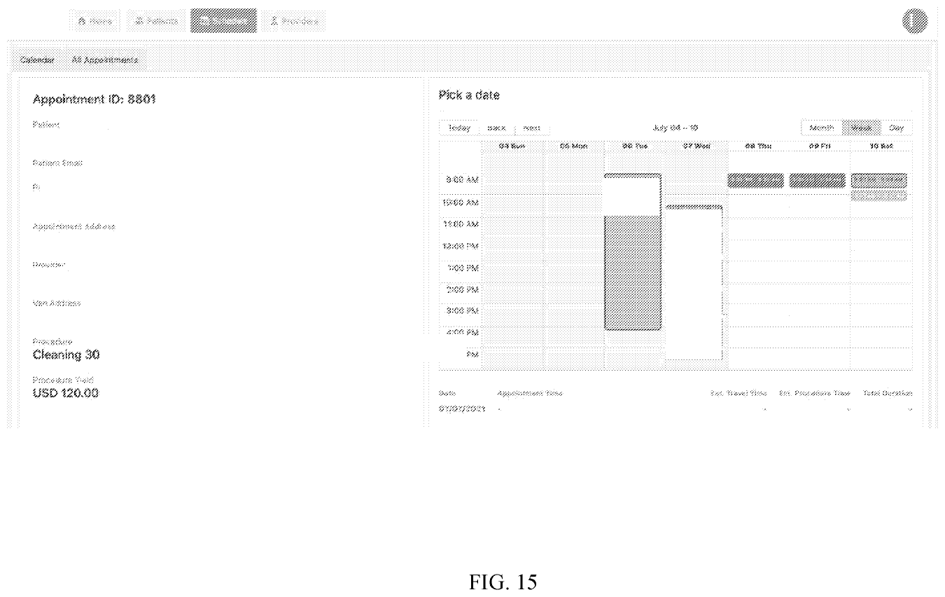

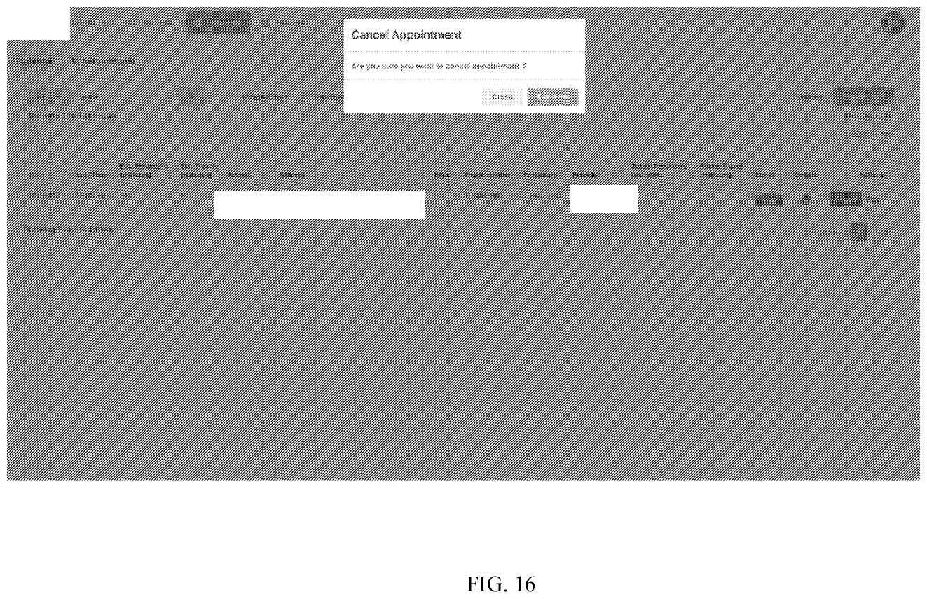

FIG. 15 illustrates an edit/rescheduling procedure view according to some embodiments of the present disclosure. Procedures may be edited via the schedule tab list view. Edits to procedures may include rescheduling of date and time options. FIG. 16 illustrates a cancel procedure view according to some embodiments of the present disclosure. Procedures may be canceled via the schedule tab list view. Procedure cancellations may be stored (e.g., in local, or remote memory) and may be filtered out of the schedule tab list view, if desired.

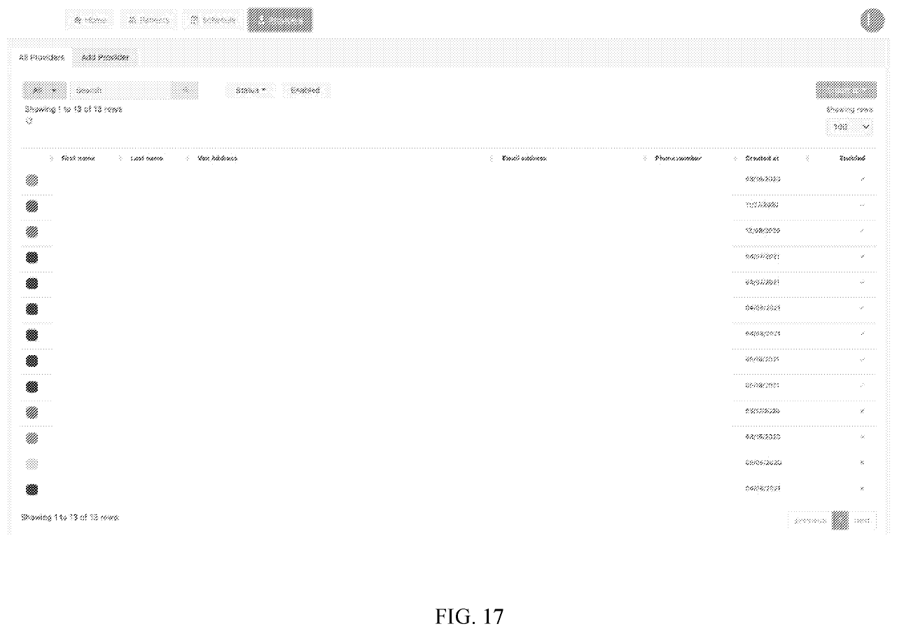

FIG. 17 illustrates a providers tab, such as the providers tab from the home screen dashboard of FIG. 11. The Super Administrator may create provider profiles. Providers may be required to pass a due diligence process. The due diligence process may be executed by one or more individuals entrusted with such a responsibility (e.g., executives at a company). Provider profiles may include the following parameters: Personal Information, Home Address, Mobile Unit Primary Van Location Address, Procedures, Duration of Procedures, Value of Procedures, Security Password, Provider Preferred User Color, and Serviceable Areas. The Personal Information parameter may further include: First Name, Last Name, Email, and Phone Number.

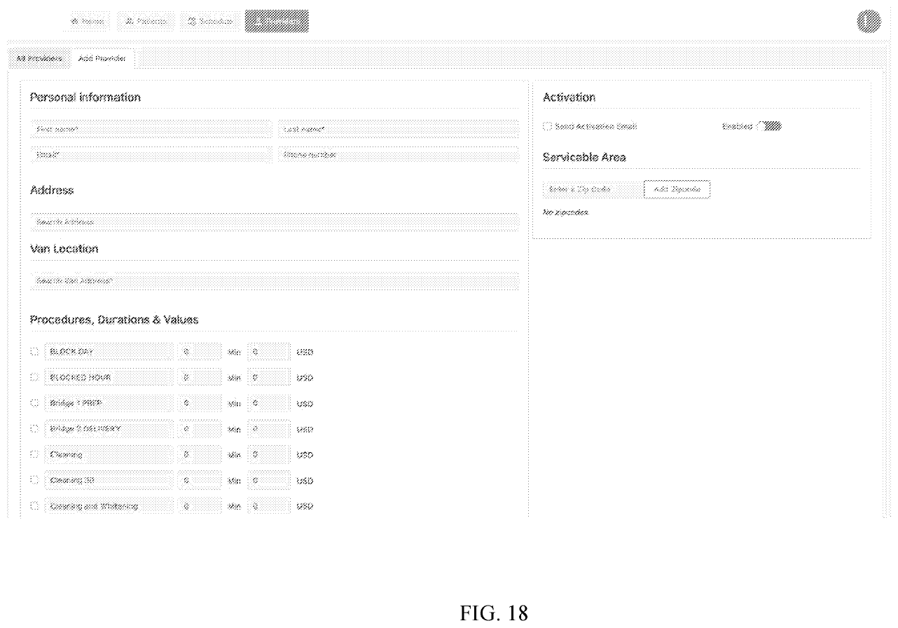

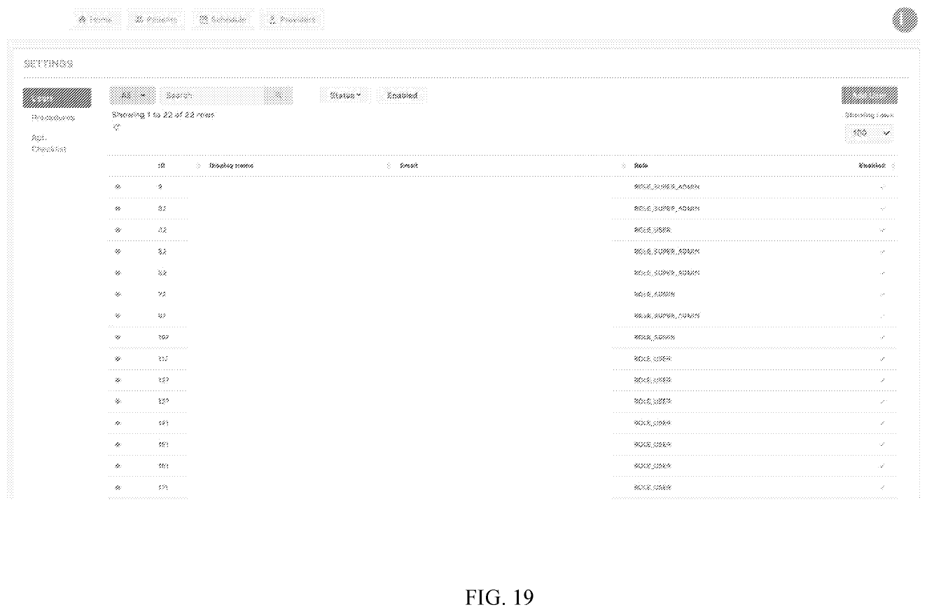

FIG. 18 illustrates an add-provider view according to some embodiments of the present disclosure. FIG. 19 illustrates a user-list view according to some embodiments of the present disclosure. User profiles may be created by a Super Administrator. Users may have access to a front-end web application for some embodiments of the present disclosure. Therefore, Users may have access to the home tab, and the schedulers tab discussed earlier herein. The following parameters may be required for a User profile to be considered active: First Name, Last Name, Email, Last Seen, Role, and Account Status.

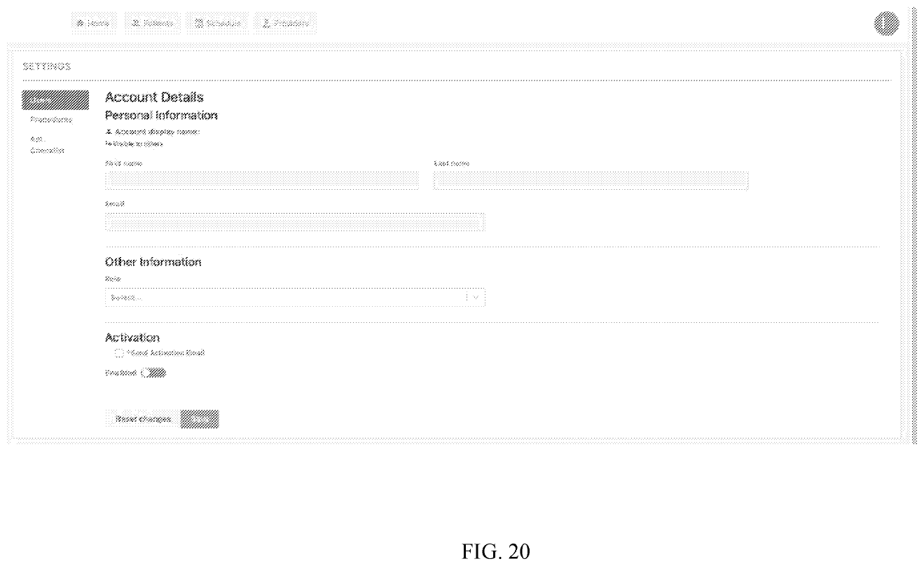

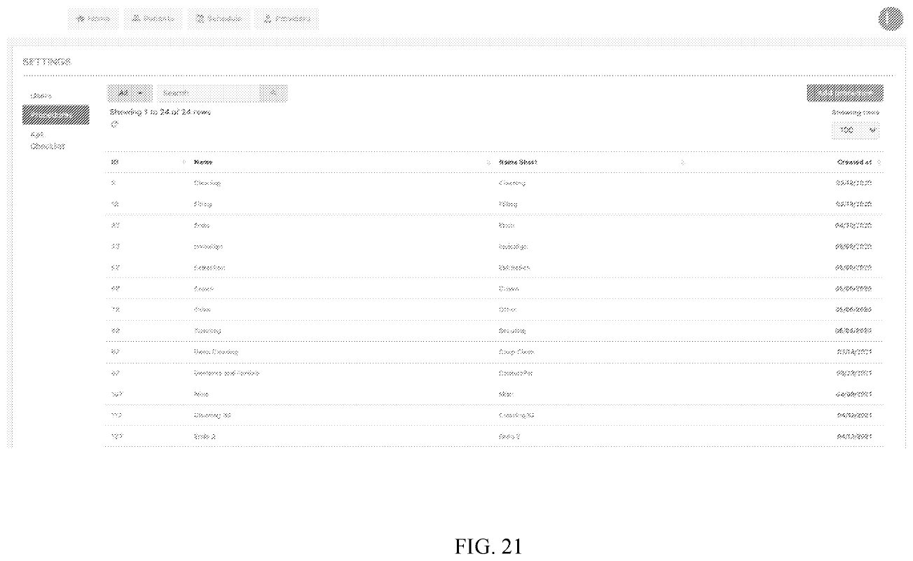

FIG. 20 illustrates an add-user dashboard according to some embodiments of the present disclosure. FIG. 21 illustrates a procedure-list view generated in accordance with some embodiments of the present disclosure. Providers may complete a procedures section when setting up their respective profiles. Procedure information may be an important part of a scheduling system (such as the scheduling system 104) because they may be used in the P.P.D.M. algorithm. Super Administrators and Admins (providers) may have the ability to access the procedure list view within the scheduler. The procedure information can be viewed and created via a Settings option located within a profile avatar.

It should be noted that while the procedures shown in FIG. 21, and other example embodiments illustrated herein, may be specific to the dental industry, systems, methods, and media disclosed herein may be applicable to a wide variety of industries where it is beneficial to optimize scheduling of procedures between providers and clients. For example, systems, methods, and media disclosed herein for scheduling procedures may be applicable to masseuses, barbers, hair stylists, nail salons, doctors, veterinarians, lawyers, technology support, repairpersons, mechanics, or any other type of service industry that may require a procedure.

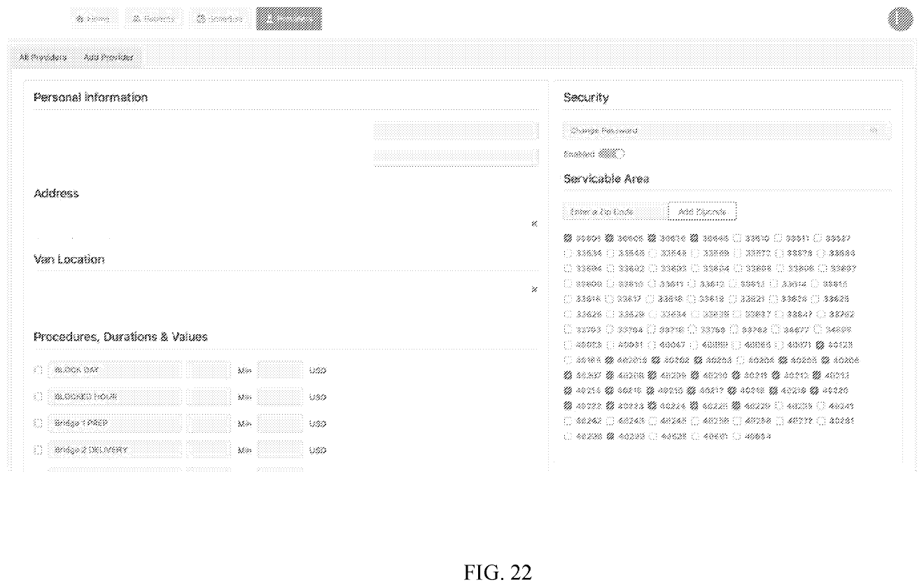

FIG. 22 illustrates a serviceable-areas view generated in accordance with some embodiments of the present disclosure. Serviceable areas may be geographical ranges within a provider's designated market. The serviceable areas view may allow providers to input parameters including information about their designated market. If a user attempts to create a procedure with a provider that is not within the serviceable area of the provider, then some embodiments of the present disclosure may request an override action to be performed. Utilizing the override action may add the serviceable area to the provider's profile. Further, utilizing the override action may add the serviceable area to the provider's profile permanently. While zip codes are used in the present embodiment as serviceable areas, other numbers or metrics are contemplated for use to compartmentalize serviceable areas.

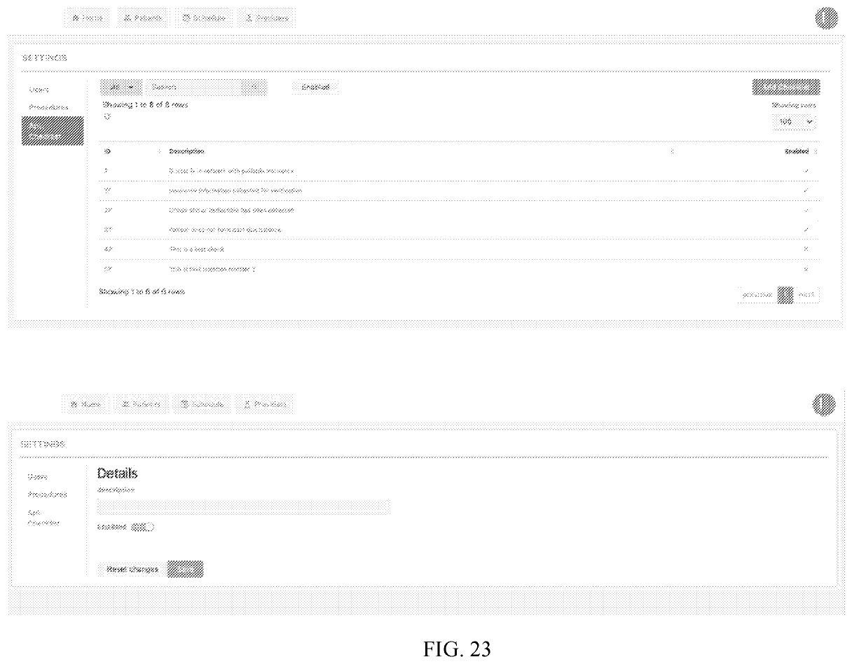

FIG. 23 illustrates a procedure-checklist view generated in accordance with some embodiments of the present disclosure. Each procedure scheduled using mechanisms disclosed herein may be paired with a procedure checklist feature. The procedure-checklist features may increase the accuracy of needed tasks to ensure that procedures are scheduled properly. Procedure checklists may be universal (e.g., the same) for each type of procedure. Super Administrators may be the only type of users that can create, update, and/or delete procedure checklists.

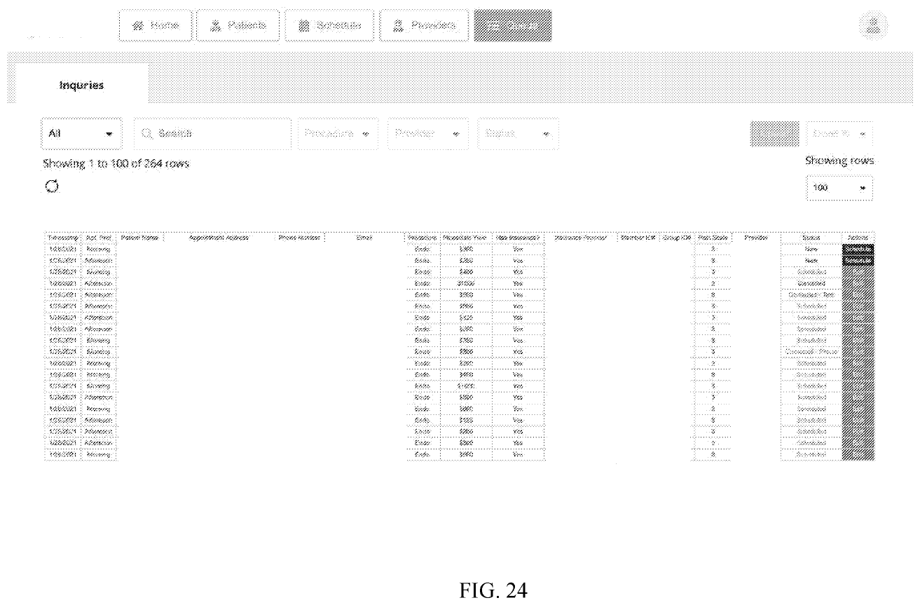

FIG. 24 illustrates a queue-list view generated in accordance with some embodiments of the present disclosure. The queue list view may be navigated to by a user via a queue tab on the home screen dashboard. The queue list view may show a list of scheduling requests that have been triggered by potential patients who have requested a procedure. Each scheduling request may allow users, administrators, and super administrators an opportunity to view any and all scheduling requests. The opportunity to view any and all scheduling requests may be an automated process via API endpoint triggers. Parameters required to successfully enter data within a queue may include: First Name, Last Name, Phone Number, Email Address (if applicable), Gender, Procedure Address, Zip Code, Date of Birth (D.O.B.), Procedure Preference Time, Chief Complaint, Insurance Company, Insurance Member ID, Insurance Group ID, Dental History, and Medical History.

An example embodiment of the present disclosure includes a mobile application on a mobile device. The mobile application can be accessed by two types of users (e.g., the super administrator and the providers). The primary users for the mobile application may be dentists (or other service providers) and their assistants. Accounts for the service providers and/or their assistants may be created in a dashboard (e.g., the dashboard of FIG. 11) by a super administrator. Further, the accounts may be activated with an email activation (e.g., a provider, or assistant, may receive a link, via email, to click in order to activate the account).

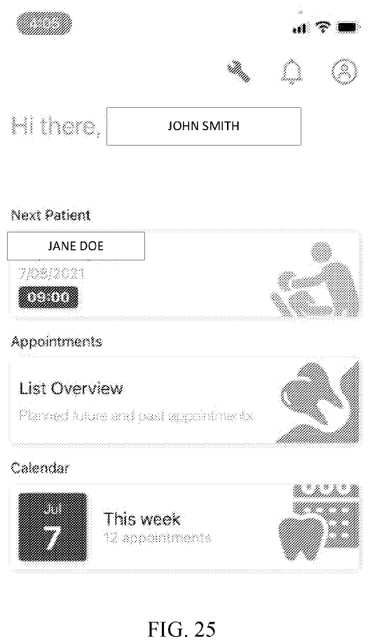

FIG. 25 illustrates a home screen for a mobile application according to some embodiments of the present disclosure. If a user has an account, the user may login to their account on the mobile application by entering their email, entering their password (while viewing or hiding the password), and then by clicking a sign in button. If a user forgot their password, the user can request a password reset using a forgot password button.

The home screen may have access to functionalities of the mobile application. For example, with respect to FIG. 25, some functionalities of the mobile application can include: Next Client or Patient (e.g., details of the next procedure), Procedures (e.g., a list of all of the scheduled procedures), Calendar (e.g., a calendar view with all of the scheduled procedures), Notifications (e.g., a list of notifications), and Profile (e.g., information from a user's profile).

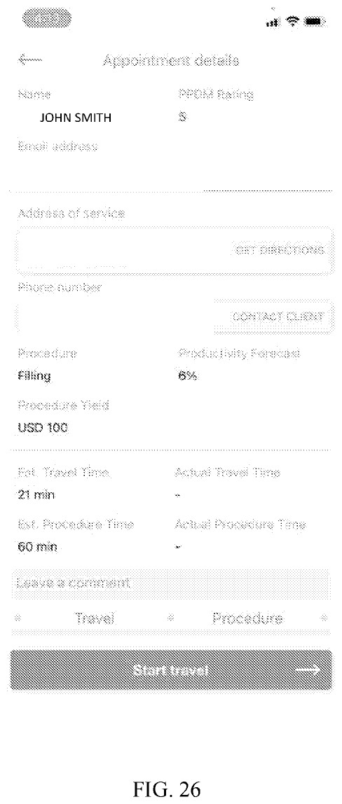

FIG. 26 illustrates a procedure-detail view for a mobile application in accordance with some embodiments of the present disclosure. The Next Patient functionality can give a brief description of a procedure in the home screen. The brief description can include: Patient's Name, Procedure Time, Procedure Date. The procedure detail view, with respect to the next patient, can provide information including: Patient's Name, Address of Service, Patient's Phone Number, Estimated Travel Time and Procedure Time, and Actual Travel Time and Procedure Time.

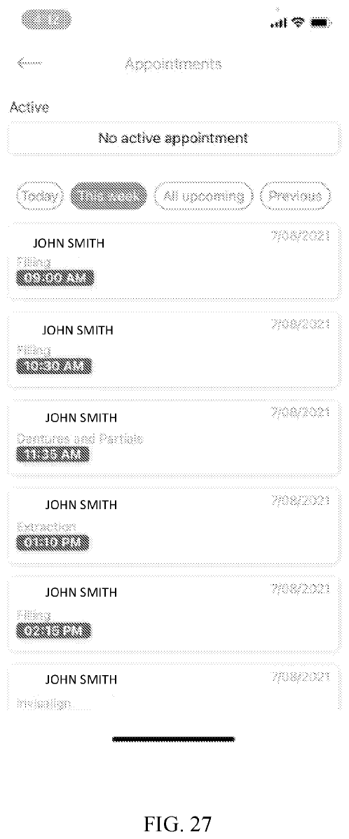

FIG. 27 illustrates a procedure-list view for a mobile application according to some embodiments of the present disclosure. The procedure-list view may be a list of all of the procedures for a provider or a user. A user may only have access to procedures that are assigned to them. The procedure list view may be sorted. An active filter on the procedure list view may include the current procedure with information about the patient's name, procedure type, procedure yield, procedure forecast, procedure time, and date. Tabs on the procedure list view may include: Today (e.g., all procedures scheduled for the current day), This Week (e.g., all procedures for the current week), All Upcoming (e.g., all upcoming procedures), and Previous (e.g., all previous procedures). The Previous tab may further include: Cancelled (e.g., all procedures that have been cancelled). Further, rescheduled procedures may be available within the corresponding (Today, This Week, or Upcoming) tab.

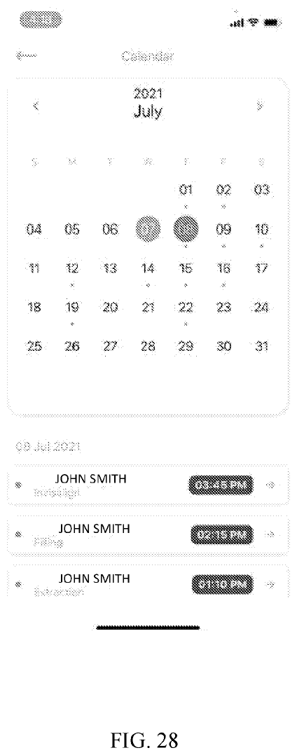

FIG. 28 illustrates a calendar view for a mobile application according to some embodiments of the present disclosure. The calendar view may include an overall view of procedures scheduled per month. Clicking a specific date may give an overview below all of the procedures scheduled on that day, displaying: Patient's Name, Procedure, and Procedure Team. If a procedure is cancelled, then the procedure will be unavailable on (e.g., removed from) the calendar view. If a procedure is rescheduled, then the procedure will be available on the date that it was rescheduled.

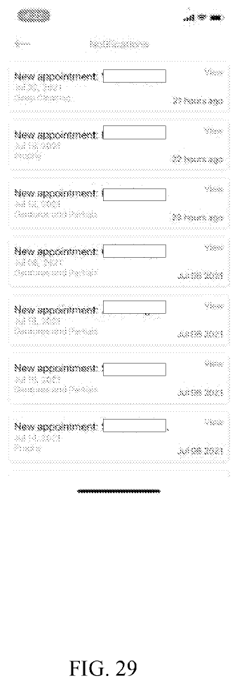

FIG. 29 illustrates a notifications view for a mobile application according to some embodiments of the present disclosure. The notification view may include an overview of all related activity to a procedure. For example, the notifications view may include: New Procedures (e.g., a new procedure may be viewed by clicking on any of the procedure cards), and Rescheduled Procedures (e.g., a rescheduled procedure may be viewed by clicking on any of the rescheduled procedure cards). Cancelled Procedures may not be clickable within the notification view.

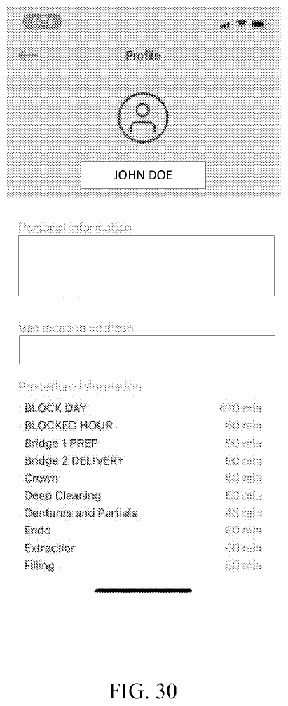

FIG. 30 illustrates a profile view for a mobile application according to some embodiments of the present disclosure. The profile view may provide an overview of a user's information. For example, the profile view may provide information that includes: First and last name of the user (e.g., a provider), address (e.g., the user's home address), email address, phone number, van location address and zip code, procedure information (e.g., procedure type, and procedure estimated time), current version of the mobile application, change password functionality, and sign out functionality.

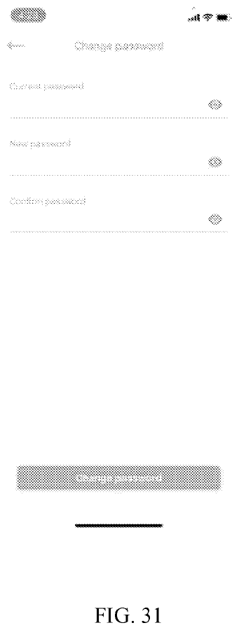

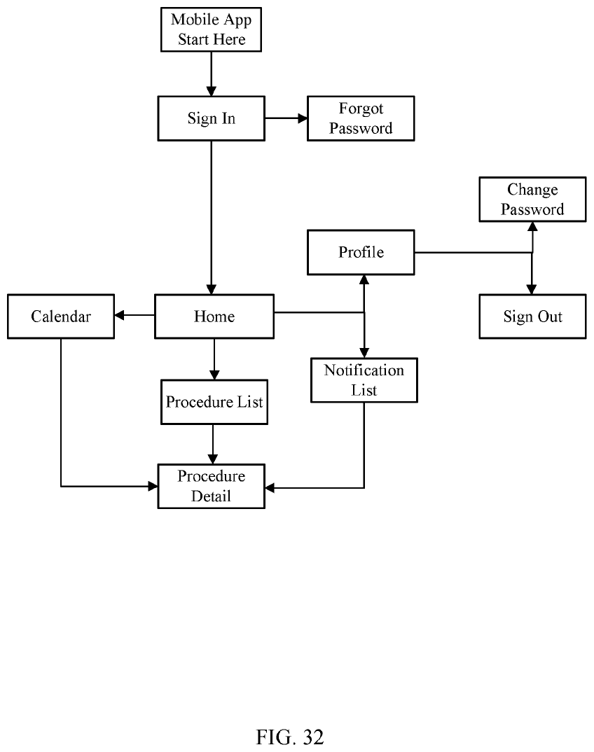

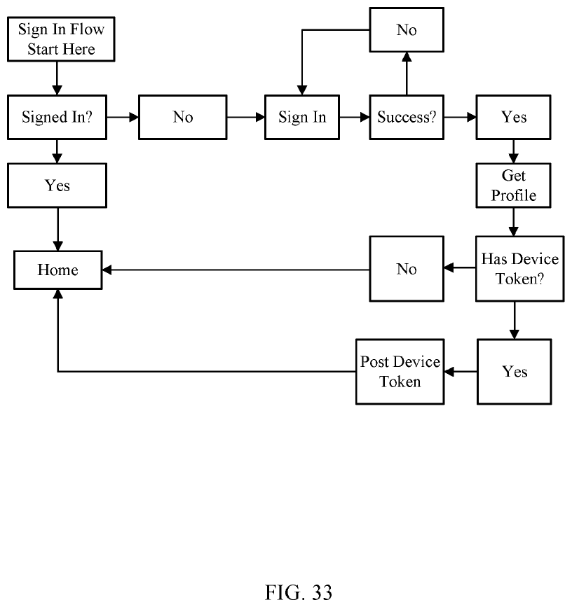

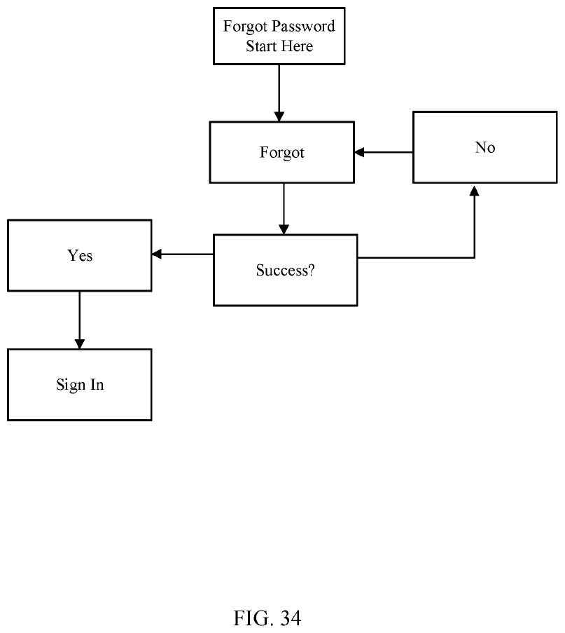

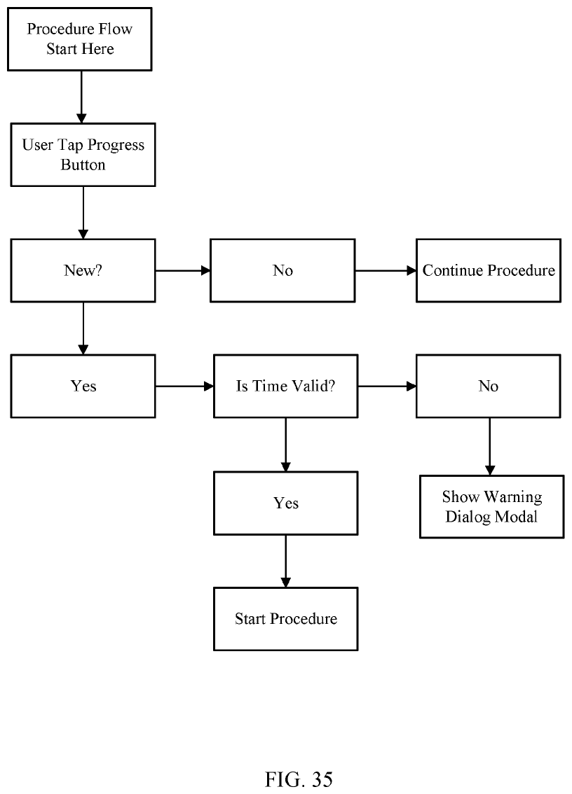

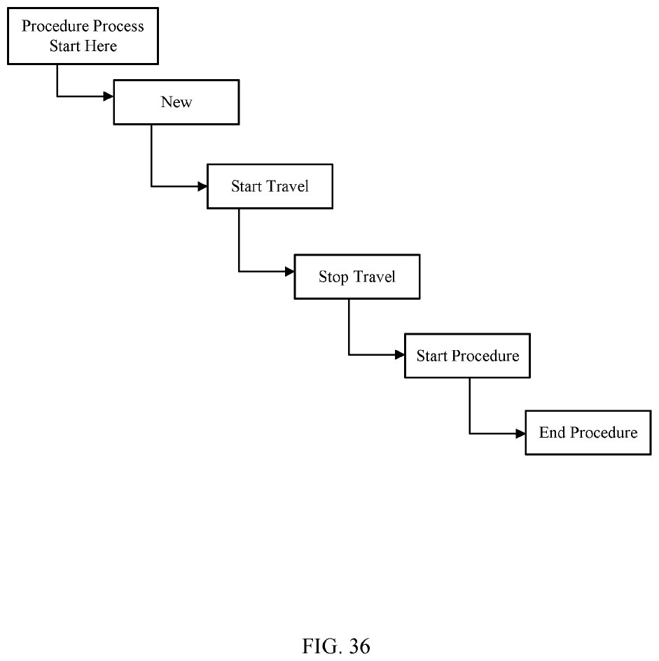

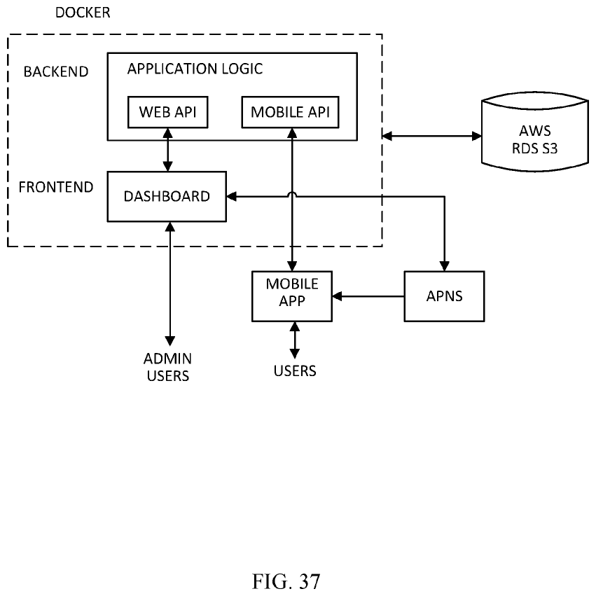

FIG. 31 illustrates a change password view for a mobile application according to some embodiments of the present disclosure. The change password view may allow a user to change their password. FIG. 32 illustrates a flow chart of a mobile application according to some embodiments of the present disclosure. FIG. 33 illustrates a flow chart of a sign-in process according to some embodiments of the present disclosure. FIG. 34 illustrates a flow chart of a forgot-password process according to some embodiments of the present disclosure. FIG. 35 illustrates a flow chart for scheduling a procedure according to some embodiments of the present disclosure. FIG. 36 illustrates a flow chart for a procedure process according to some embodiments of the present disclosure. FIG. 37 illustrates a flow chart for a mobile application architecture according to some embodiments of the present disclosure.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

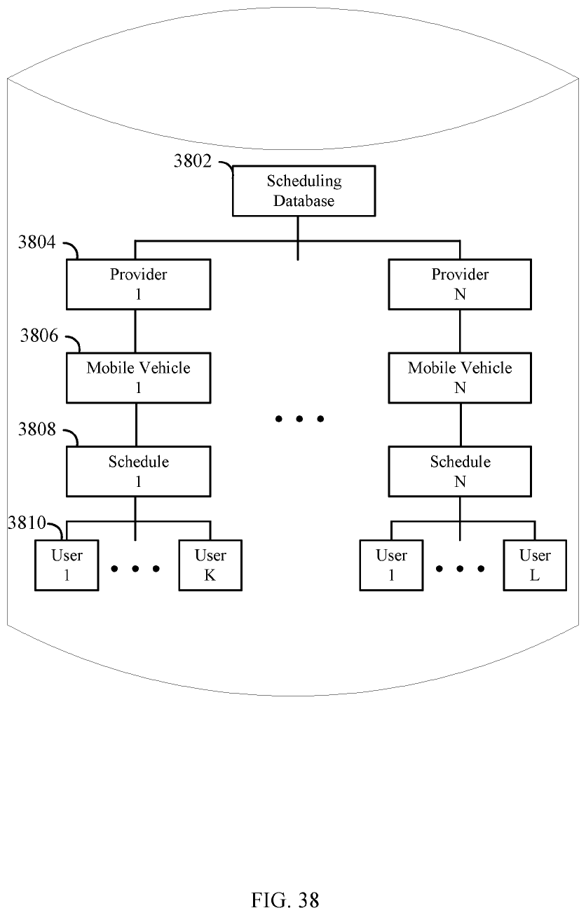

FIG. 38 illustrates a data relationship of a scheduling database according to some embodiments of the present disclosure. In some examples, memory 210 of computing device 110 and/or memory 220 of server 120 in FIG. 2 can include the scheduling database 3802. The scheduling database 3802 can include one or more providers 3804. Each provider 3804 can correspond to a mobile vehicle 3806. Thus, the one or more providers 3804 can correspond to one or more mobile vehicles 3806. However, in some examples, a provider 3804 can be mapped to multiple mobile vehicles 3806. In further examples, each provider 3804 can correspond to a schedule 3808. Thus, the one or more providers 3804 can correspond to one or more schedules 3808. In even further examples, each schedule 3808 can correspond to one or more user accounts 3810. Thus, multiple user accounts can access a schedule 3808 to generate one or more procedures for a mobile vehicle moving and perform procedures corresponding to procedures.

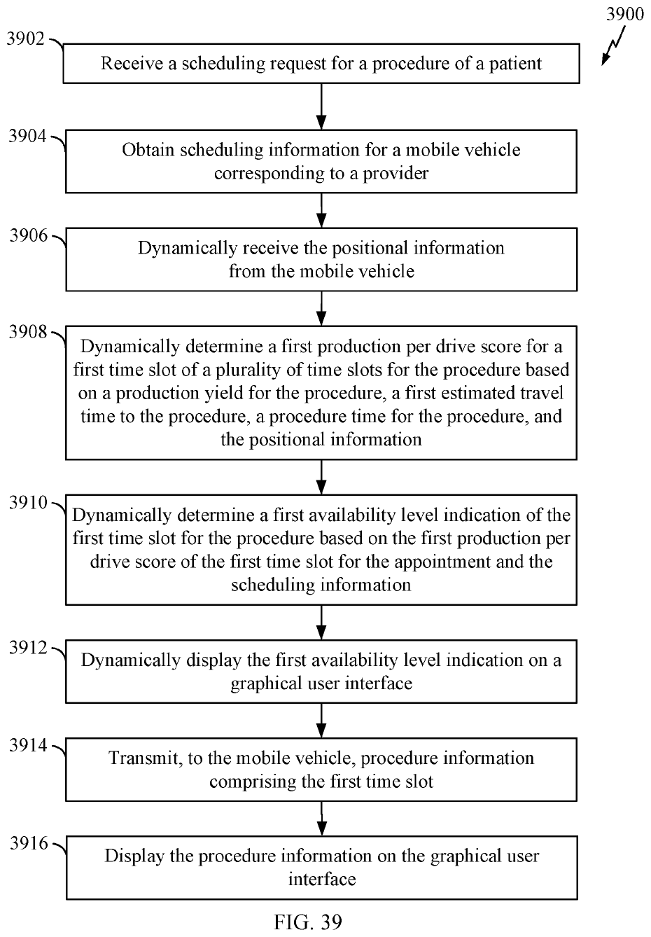

FIG. 39 illustrates a flow chart for using positional information for a mobile vehicle to generate dynamic scheduling according to some embodiments of the present disclosure. In some examples, process 3900 may be carried out by an apparatus or a system. The apparatus or the system can be the server(s) 120 and/or the computing device(s) 110 illustrated in FIG. 3, e.g., employing circuitry and/or software configured according to the block diagram illustrated in FIGS. 1 and 2. It should be appreciated that process 3900 may be carried out by any suitable means for carrying out the functions or algorithm described below. In some examples, any suitable system can be used to implement process 3900. Additionally, although the blocks of the flowchart are presented in a sequential manner, in some examples, one or more of the blocks may be performed in a different order than presented, in parallel with another block, or bypassed.

At block 3902, the apparatus can receive a scheduling request for a procedure of a patient. In some examples, the scheduling request can include at least one of a service location of the procedure or a procedure indication of the procedure. In some examples, the procedure is configured to occur inside of, in proximity to, or in connection with the mobile vehicle. In further examples, the service location of the procedure can include an address, a geographic coordinate, or any suitable location indication at which the procedure is configured to occur. In further examples, the procedure indication of the procedure can indicate any suitable procedure (e.g., cleaning, filling, endo, Invisalign, extraction, crown, scouting, deep cleaning, dentures and partials, etc.). For example, a user (e.g., patient) can want to schedule a dental procedure near the house. The user can provide the scheduling request including a service location (e.g., a public parking lot address near patient's house) and/or a procedure indication (e.g., cleaning) that the patient wants to receive (e.g., by giving a call to the apparatus or the system, or accessing a website or an application on a mobile device).

At block 3904, the apparatus can obtain scheduling information for a mobile vehicle corresponding to a preferred provider. In some examples, a provider is a person who is able to provide services to a customer/patient and bill for services. In further examples, providers can register procedures or services that other providers can perform (e.g., on the apparatus). Each provider may have personalized procedure times, procedure production, and daily production goals that are assigned. In further examples, the apparatus can display multiple providers based on a profile of the patient. For example, the profile of the patient can include insurance information associated with the patient. Thus, the multiple providers displayed may only be providers who accept the insurance of the patient. In further examples, the profile of the patient can include the patient's address. In some examples, the apparatus can receive information for the multiple providers, and the information for the multiple providers can include service area indications corresponding to the multiple providers. Thus, the multiple providers displayed can be providers located within a predetermined or configurable distance from the patient's address. Then, the user can select a preferred provider among the multiple providers displayed, and the apparatus can receive an input indicative of the preferred provider.

In further examples, the scheduling information for the mobile vehicle can correspond to the preferred provider. For example, the scheduling information for the mobile vehicle can include procedures that the mobile vehicle is configured to perform at different locations. In some examples, one provider may correspond with one mobile vehicle. However, it should be appreciated that one provider can correspond to more than one mobile vehicle to simultaneously perform procedures at different locations.

At block 3906, the apparatus can dynamically receive the positional information from the mobile vehicle. In some examples, the positional information can include a current location of the mobile vehicle. In further examples, the mobile vehicle can dynamically or in near real-time transmit the positional information using a global positioning satellite receiver, a network location system, a Wi-Fi positioning system, or any other suitable location sensor or system. Then, the apparatus can dynamically or in near real-time receive the positional information from the mobile vehicle.

At block 3908, the apparatus can dynamically determine a first production per drive score for a first time slot of multiple time slots for the procedure based on a production yield for the procedure, a first estimated travel time to the procedure, a procedure time for the procedure, and the positional information. In some examples, the apparatus can determine the production yield and the procedure time based on the procedure indication of the procedure from the scheduling request. For example, when the apparatus receives a request including a procedure (e.g., cleaning), the apparatus can determine its procedure time (e.g., determined based on an average time for the cleaning or a time provided by the provider) and its production yield (e.g., determined based on the amount that an insurance company determines or that the provider provides for the cleaning). In some examples, a time slot (e.g., the first time slot) can be determined based on a procedure time (e.g., a time period to perform a procedure inside of, in proximity to, or in connection with the mobile vehicle). In further examples, a time slot can be a procedure time and any other suitable time (e.g., preparation time and cleaning time after the procedure of the procedure). In some examples, the first estimated travel time is determined based on a distance from the current location of the positional information to the service location. In other examples, the first estimated travel time is determined based on a distance from a previous location to the service location. In some examples, the first estimated travel time is determined based on the remaining time period to the first time slot. For example, when the remaining time period from the current time to the first time slot is equal to or shorter than a predetermined time period (e.g., 30 minutes, 1 hour, 2 hours, etc.), the first estimated travel time is determined based on a distance from the current location of the positional information to the service location. In further examples, the first production per drive score is determined by:

$$the first production per drive score = \frac{the production yield for the appointment}{( the first estimated travel time + the procedure time )} .$$

In further examples, the apparatus can dynamically determine a second production per drive score for a second time slot of the multiple time slots for the procedure based on the production yield for the procedure, a second estimated travel time to the procedure, and the procedure time for the procedure. In some examples, the apparatus does not need to consider the positional information for the second production per drive score. For example, when the remaining time period from the current time to the second time slot is equal to or longer than a predetermined time period (e.g., 30 minutes, 1 hour, 2 hours, etc.), the apparatus does not consider the positional information to determine the production per drive score. In some examples, the apparatus can determine the second estimated travel time based on a previous location of a previous procedure and the service location of the procedure. In further examples, the second production per drive score is determined by:

$$the second production per drive score = \frac{the production yield for the appointment}{( the second estimated travel time + the procedure time )} .$$

In even further examples, the apparatus can determine an estimated travel time to the procedure based on a base station (e.g., a center of a serviceable zone, a driver's house, any suitable location to place the mobile vehicle when the mobile vehicle does not travel for an procedure) when a time period between the time slot and a previous time slot is more than a predetermined threshold. For example, when a time slot for the procedure is 4:00 PM and a previous time slot for a previous procedure is 10:00 AM, the apparatus can determine the estimated travel time to the procedure from the base station to the service location of the procedure. However, when another procedure is made at 2:00 PM, the apparatus can dynamically determine the estimated time travel to the procedure from the location of another procedure at 2:00 PM to the service location of the procedure at 4:00 PM.

At block 3910, the apparatus can dynamically determine a first availability level indication of the first time slot for the procedure based on the first production per drive score of the first time slot for the procedure and the scheduling information. In some examples, to determine the first availability level indication of the first time slot, the apparatus can calculate an availability score by the first production per drive score divided by a production per unit time score and determine the first availability level indication by comparing the availability score with one or more thresholds. For example, the availability score can be defined by:

$$\frac{the first production per drive score}{a production per unit time score} \times 100 .$$

In some examples, the production per unit time score can be calculated by the provider's daily production goal divided by daily availability. In further examples, the first availability level indication of the first time slot can be indicated as one of multiple groups. For example, group 1 of the first availability level indication can be the availability score of lower than a first threshold (e.g., 50), group 2 of the first availability level indication can be the availability score between the first threshold and the second threshold (e.g., 75), and group 3 of the first availability level indication can be the availability score of equal to or higher than the second threshold (e.g., 75). It should be appreciated that the first and second thresholds can be configurable or predetermined. In further examples, the number of thresholds to classify groups depending on availability scores is not limited to 3 but could be any suitable number of groups.

In further examples, the apparatus can dynamically determine a second availability level indication of the second time slot for the procedure based on the second production per drive score of the second time slot for the procedure. In some examples, the second availability level indication of the second time slot for the procedure is further determined based on the second estimated travel time. In further examples, the second time slot (e.g., 1:00-2:00 PM) for the procedure may be within the second estimated travel time (e.g., 50 minutes) after a previous time slot (12:00-12:30 PM) for the previous procedure. In even further examples, the second time slot (e.g., 1:00-2:00 PM) for the procedure may be within a dynamic travel time (e.g., 50 minutes) to travel from the current location (e.g., at 12:30 PM) of the positional information to the service location. In the examples, in response to the second time slot for the procedure being within the second estimated travel time or the dynamic travel time, the second availability level indication of the second time slot indicates unavailability for the procedure. For example, the previous time slot for the previous procedure (e.g., the end of the previous time slot) and the second time slot (e.g., the beginning of the second time slot) for the procedure are 1:00 PM and 1:30 PM.

When the second estimated travel time to travel from the service location of the previous procedure to the service location of the procedure is 30 minutes, the apparatus determines that the second time slot for the procedure is unavailable. In another example, the apparatus can dynamically, or in near real-time, determine that the second time slot for the procedure is unavailable based on the current location of the mobile vehicle. For example, the previous time slot for the previous procedure (e.g., the end of the previous time slot) and the second time slot (e.g., the beginning of the second time slot) for the procedure are 1:00 PM and 2:00 PM. When the second estimated travel time to travel from the service location of the previous procedure to the service location of the procedure is 30 minutes, the second time slot should be available. However, when the dynamic travel time to travel from the current location of the positional information to the service location is 1 hour, the apparatus can determine that the second availability level indication of the second time slot indicates unavailability for the procedure. In further examples, when the production per drive score for a time slot is lower than a predetermined and configurable score, the apparatus can determine that the time slot for the procedure is unavailable.

At block 3912, the apparatus can dynamically display the first availability level indication and/or the second availability level indication on a graphical user interface. In some examples, the first availability level indication and/or the second availability level indication is displayed using a color corresponding to the first availability level indication. In the example above, the apparatus can display group 1, group 2, and group 3 of the availability level indication as Red, Orange, and Green, respectively. It should be appreciated that the color of the availability level indication is not limited to Red, Orange, and Green. The availability level indication can be displayed with any suitable color, symbol, or indication.

At block 3914, the apparatus can dynamically transmit, to the mobile vehicle, procedure information including the first time slot. In some examples, the user can select the first time slot for the procedure, and the apparatus can receive a user input indicative of the first time slot for the procedure. In further examples, the procedure information can further include the service location of the procedure, the procedure indication of the procedure, and/or the profile of the patient. For example, when the user selects the first time slot for the procedure, the apparatus can send the procedure information to the mobile vehicle to be ready to move to the procedure or to be aware of the procedure.

At block 3916, the apparatus can display the procedure information on the graphical user interface. In some examples, the apparatus can display a summary of the procedure made by the user. In other examples, the apparatus can also display a prior procedure for the patient. In further examples, the apparatus can display a previous or next procedure before or after the procedure for the mobile vehicle to be prepared for the procedure.

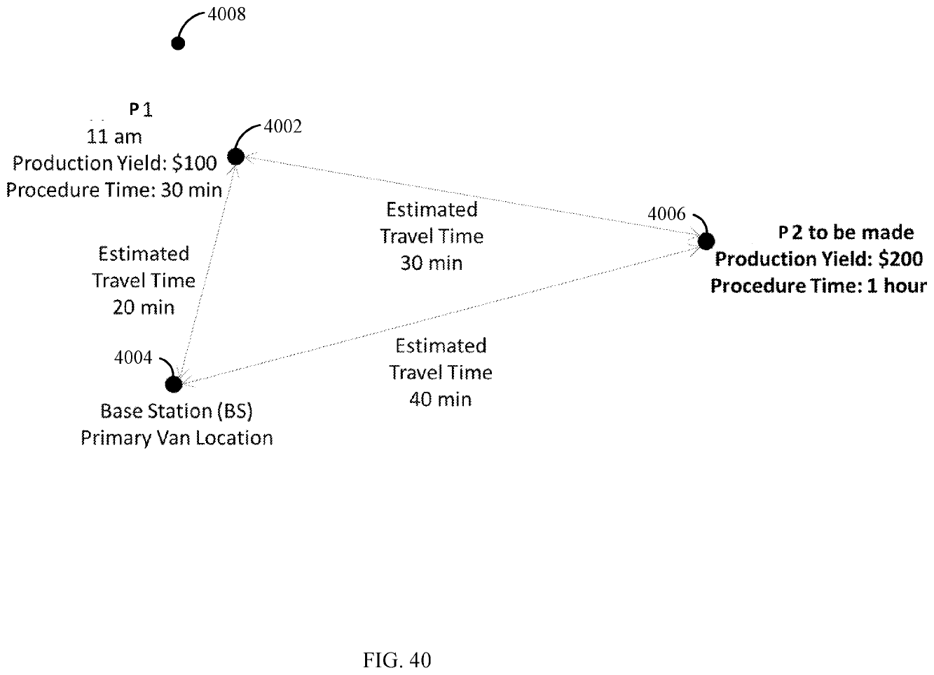

FIG. 40 illustrates an example scenario based on a scheduling algorithm according to some embodiments of the present disclosure. In the example, Procedure 1 (4002) is already entered into the system. Procedure 1 (4002) includes the production yield of $100 and the procedure time of 30 minutes. The estimated travel time from a base station 4004 to the service location of Procedure 1 (4002) is 30 minutes. A user may thereafter desire to make Procedure 2 (4006) for a procedure with a production yield of $200 and a procedure time of 60 minutes. the estimated travel times from Procedure 1 (4002) to Procedure 2 (4006) and from the base station 4004 to Procedure 2 (4006) are 30 minutes and 40 minutes, respectively.

In some examples, the apparatus can determine the P.P.D.M. score by the production yield divided by the estimated travel time from the previous location of the previous procedure (i.e., Procedure 1 (4002). In the example scenario, the P.P.D.M. score is 6.67 ($200/30 min). When the apparatus determines P.P.D.M. thresholds as 20 and 40 for the availability level indication (e.g., Red: P.P.D.M.<20, Orange: 20<=P.P.D.M. score<40, and Green: P.P.D.M.>=40), the availability level indication for Procedure 2 (4006) is displayed as red because the P.P.D.M. score is lower than 20. It should be appreciated that the P.P.D.M. thresholds are configurable, and the P.P.D.M. thresholds of 20 and 40 are mere example thresholds.

In other examples, the apparatus can determine the production per drive (P.P.D) score by the production yield/(the estimated travel time+the procedure time). In the example scenario, the P.P.D score for Procedure 2 is 2.22 ($200/(30 min+60 min)). If the provider has a daily productivity goal of $900, the provider's production goal per min (P.P.G.M.) is 2.143 (Provider's Daily Production Goal ($900)/Daily Availability (7 hours×60 min/hour)). The availability score can be defined as: P.P.D score/P.P.G.M×100. Thus, the availability score for Procedure 2 is 103.74 (2.22/2.14×100). When the apparatus determines the availability thresholds as 50 and 75 for the availability level indication (e.g., Red: P.P.D./P.P.G.M×100<50, Orange: 50<=P.P.D./P.P.G.M×100<75, and Green: P.P.D./P.P.G.M×100>=75), the availability level indication for Procedure 2 (4006) is displayed as green because the availability score is higher than 75. It should be appreciated that the availability thresholds are configurable, and the availability thresholds of 50 and 75 are mere example thresholds.

In further examples, the apparatus can determine that certain time slots for Procedure 2 (4006) are unavailable due to prior procedure(s). For example, the apparatus can display a time slot between 10 AM and 11:30 AM being unavailable because the mobile vehicle cannot perform Procedure 2 (4006) after or before Procedure 1 (4002) between 10 AM and 11:30 AM. In further examples, the apparatus can determine that certain time slots for Procedure 2 (4006) are unavailable due to the current location of the mobile vehicle. For example, the mobile vehicle is at a location 4008 at 11:30 AM, which is 40 minutes away from the service location of Procedure 2 (4006). Then, the user wants to schedule Procedure 2 (4006) at 12:00 PM. Then, the apparatus can indicate that the time slot at 12:00 PM for Procedure 2 (4006) is unavailable.

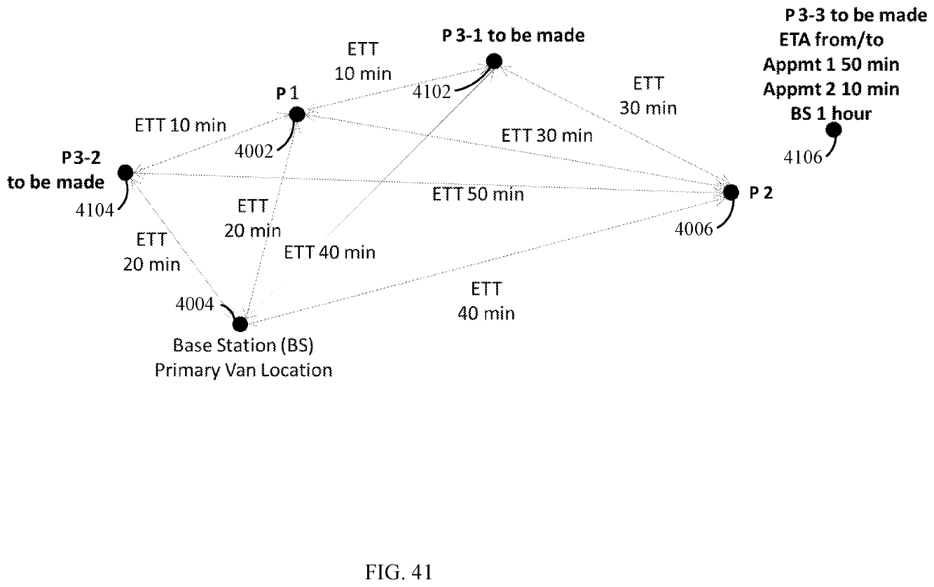

FIG. 41 illustrates another example scenarios based on a scheduling algorithm according to some embodiments of the present disclosure. In this example scenario, the base station 4004 and Procedure 1 (4002) at 11:00 AM are the same as the scenario of FIG. 40. In the example scenario, Procedure 2 (4006) at 2:00 PM is already entered into the system. Here, the user wants to schedule Procedure 3-1 (4102), Procedure 3-2 (4104), or Procedure 3-3 (4106) at a different service location. The production yield and procedure time for Procedure 3 (i.e., Procedure 3-1 (4102), Procedure 3-2 (4104), or Procedure 3-3 (4106)) are $50 and 20 minutes, respectively.

For Procedure 3-1 (4102) in some examples, the apparatus can receive a scheduling request for Procedure 3-1 (4102) at 12:00 PM or 1:00 PM. In some examples, the apparatus can determine a previous procedure as Procedure 1 (4002) because Procedure 1 (4002) is configured to be performed at 11:00 AM. The estimated travel time from Procedure 1 (4002) to Procedure 3-1 (4102) is 10 minutes. In the examples, a P.P.D.M score for Procedure 3-1 (4102) may be 5 ($50/10 min). Since the P.P.D.M. score is lower than 20, the apparatus displays the availability level indication for Procedure 3-1 (4102) as red. The availability level indication for Procedure 3-1 (4102) is the same at 12:00 PM and 1:00 PM. In other examples, the apparatus can determine the P.P.D. score as 1.67 ($50/(10 min+20 min)). When the provider has a daily productivity goal of $900, the P.P.G.M is 2.143 ($900/(7 hours×60 min/hr). Thus, the availability score for Procedure 3-1 (4102) is 77.7 (1.667/2.143×100). Since the availability score for Procedure 3-1 is higher than 75, the apparatus displays the availability level indication for Procedure 3-1 (4102) as green. The availability level indication for Procedure 3-1 (4102) is the same at 12:00 PM and 1:00 PM.

For Procedure 3-1 (4102) in further examples, the apparatus can receive a scheduling request for Procedure 3-1 (4102) at 3:00 PM. In some examples, the apparatus can determine a previous procedure as Procedure 2 (4006) because Procedure 2 (4106) is configured to be performed at 2:00 PM. The estimated travel time from Procedure 2 (4002) to Procedure 3-1 (4102) is 30 minutes. In the example, a P.P.D.M score for Procedure 3-1 (4102) as 1.667 ($50/30 min). Since the P.P.D.M. score is lower than 20, the apparatus displays the availability level indication for Procedure 3-1 (4102) as red. In other examples, the apparatus can determine the P.P.D. score as 1 ($50/(30 min+20 min). When the provider has a daily productivity goals of $900, the P.P.G.M is 2.143 ($900/(7 hours×60 min/hr). Thus, the availability score for Procedure 3-1 (4102) is 46.7 (1/2.143×100). Since the availability score for Procedure 3-1 is lower than 50, the apparatus displays the availability level indication for Procedure 3-1 (4102) as red.

For Procedure 3-2 (4104) in some examples, the apparatus can receive a scheduling request for Procedure 3-2 (4104) at 12:00 PM or 1:00 PM. In some examples, the apparatus can determine a previous procedure as Procedure 1 (4002) because Procedure 1 (4002) is configured to be performed at 11:00 AM. The estimated travel time from Procedure 1 (4002) to Procedure 3-2 (4104) is 10 minutes. In the examples, a P.P.D.M score for Procedure 3-2 (4104) as 5 ($50/10 min). Since the P.P.D.M. score is lower than 20, the apparatus displays the availability level indication for Procedure 3-2 (4104) as red. The availability level indication for Procedure 3-2 (4104) is the same at 12:00 PM and 1:00 PM. In other examples, the apparatus can determine the new P.P.D. score as 1.67 ($50/(10 min+20 min)). When the provider has a daily productivity goal of $900, the P.P.G.M is 2.143 ($900/(7 hours×60 min/hr). Thus, the availability score for Procedure 3-2 (4104) is 77.7 (1.667/2.143×100). Since the availability score for Procedure 3-2 (4104) is higher than 75, the apparatus displays the availability level indication for Procedure 3-2 (4104) as green. The availability level indication for Procedure 3-2 (4104) is the same at 12:00 PM and 1:00 PM.

For Procedure 3-2 (4104) in further examples, the apparatus can receive a scheduling request for Procedure 3-2 (4104) at 3:00 PM. In some examples, the apparatus can determine a previous procedure as Procedure 2 (4006) because Procedure 2 (4106) is configured to be performed at 2:00 PM. The estimated travel time from Procedure 2 (4002) to Procedure 3-2 (4104) is 50 minutes. In the example, a P.P.D.M score for Procedure 3-2 (4104) as 1 ($50/50 min). Since the P.P.D.M. score is lower than 20, the apparatus displays the availability level indication for Procedure 3-2 (4104) as red. In other examples, the apparatus can determine the new P.P.D. score as 0.714 ($50/(50 min+20 min)). When the provider has a daily productivity goal of $900, the P.P.G.M is 2.143 ($900/(7 hours×60 min/hr). Thus, the availability score for Procedure 3-2 (4104) is 33.3 (0.714/2.143×100). Since the availability score for Procedure 3-2 (4104) is lower than 50, the apparatus displays the availability level indication for Procedure 3-2 (4104) as red. In further examples, the apparatus can display Procedure 3-2 (4104) at 3:00 PM as unavailable because the mobile vehicle can leave the service location of Procedure 2 (4006) at 2:20 PM and cannot arrive at the service location of Procedure 3-2 (4104) by 3:00 PM.

For Procedure 3-3 (4106) in some examples, the apparatus can receive a scheduling request for Procedure 3-3 (4106) at 12:00 PM or 1:00 PM. In some examples, the apparatus can determine a previous procedure as Procedure 1 (4002) because Procedure 1 (4002) is configured to be performed at 11:00 AM. The estimated travel time from Procedure 1 (4002) to Procedure 3-3 (4106) is 50 minutes. In the example, a P.P.D.M score for Procedure 3-3 (4106) is 1 ($50/50 min). Since the P.P.D.M. score is lower than 20, the apparatus displays the availability level indication for Procedure 3-3 (4106) as red. The availability level indication for Procedure 3-3 (4106) is the same at 12:00 PM and 1:00 PM. In other examples, the apparatus can determine the new P.P.D. score as 0.714 ($50/(50 min+20 min)). When the provider has a daily productivity goal of $900, the P.P.G.M score is 2.143 ($900/(7 hours×60 min/hr). Thus, the availability score for Procedure 3-3 (4106) is 33.3 (0.714/2.143×100). Since the availability score for Procedure 3-3 (4106) is lower than 50, the apparatus displays the availability level indication for Procedure 3-3 (4106) as red. The availability level indication for Procedure 3-3 (4106) is the same at 12:00 PM and 1:00 PM. In further examples, the apparatus can display Procedure 3-3 (4106) at 12:00 PM is unavailable because the mobile vehicle can leave the service location of Procedure 1 (4006) at 11:30 AM and can arrive at the service location of Procedure 3-3 (4106) at 12:20 PM, which is later than the time slot for Procedure 3-3 (4106).

For Procedure 3-3 (4106) in further examples, the apparatus can receive a scheduling request for Procedure 3-3 (4106) at 3:00 PM. In some examples, the apparatus can determine a previous procedure as Procedure 2 (4006) because Procedure 2 (4106) is configured to be performed at 2:00 PM. The estimated travel time from Procedure 2 (4002) to Procedure 3-3 (4106) is 10 minutes. In the example, a P.P.D.M score for Procedure 3-3 (4106) as 5 ($50/10 min). Since the P.P.D.M. score is lower than 20, the apparatus displays the availability level indication for Procedure 3-3 (4106) as red. In other examples, the apparatus can determine the P.P.D. score as 01.667 ($50/(10 min+20 min). When the provider has a daily productivity goal of $900, the P.P.G.M is 2.143 ($900/(7 hours×60 min/hr). Thus, the availability score for Procedure 3-3 (4106) is 77.7 (1.667/2.143×100). Since the availability score for Procedure 3-3 (4106) is higher than 75, the apparatus displays the availability level indication for Procedure 3-3 (4106) as green.

It should be noted that, as used herein, the term mechanism can encompass hardware, software, firmware, or any suitable combination thereof. It should be understood that the above-described steps of any processes or flowcharts disclosed herein can be executed or performed in any order or sequence not limited to the order and sequence shown and described in the figures. Also, some of the above steps of the processes or flowcharts can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. A method for using positional information for a mobile vehicle to generate dynamic scheduling, comprising steps of:
receiving a scheduling request for a procedure of a patient, the procedure configured to occur inside of, in proximity to, or in connection with the mobile vehicle;
obtaining scheduling information for the mobile vehicle corresponding to a preferred provider;
dynamically receiving the positional information from the mobile vehicle, the positional information comprising a current location of the mobile vehicle;
determining a first production per drive score for a first time slot of a plurality of time slots for the procedure based on a production yield for the procedure, a first estimated travel time to the procedure, a procedure time for the procedure, and the positional information;
determining a first availability level indication of the first time slot for the procedure based on the first production per drive score of the first time slot for the procedure and the scheduling information;
dynamically displaying the first availability level indication on a graphical user interface;
dynamically transmitting, to the mobile vehicle, procedure information comprising the first time slot; and
displaying the procedure information on the graphical user interface.

2. The method of claim 1, wherein the scheduling request comprises a service location of the procedure,
wherein the first estimated travel time is determined based on a distance from the current location of the positional information to the service location, and
wherein the procedure information further comprises the service location.

3. The method of claim 1, wherein the first production per drive score is determined by:
$$the first production per drive score = \frac{the production yield for the appointment}{( the first estimated travel time + the procedure time )} .$$

4. The method of claim 3, wherein determining the first availability level indication of the first time slot comprises the steps of:
calculating an availability score by the first production per drive score divided by a production per unit time score; and
determining the first availability level indication by comparing the availability score with one or more thresholds.

5. The method of claim 1, wherein the first availability level indication is displayed using a color corresponding to the first availability level indication.

6. The method of claim 1, further comprising:
dynamically determining a second production per drive score for a second time slot of the plurality of time slots for the procedure based on the production yield for the procedure, a second estimated travel time to the procedure, and the procedure time for the procedure;
dynamically determining a second availability level indication of the second time slot for the procedure based on the second production per drive score of the second time slot for the procedure; and
dynamically displaying the second availability level indication on a graphical user interface,
wherein the scheduling request comprises a service location of the procedure, and
wherein the second estimated travel time is determined based on a previous location of a previous procedure and the service location of the procedure.

7. The method of claim 6, wherein the second availability level indication of the second time slot for the procedure is further determined based on the second estimated travel time,
wherein in response to the second time slot for the procedure being within the second estimated travel time after a previous time slot for the previous procedure or a dynamic travel time to travel from the current location of the positional information to the service location, the second availability level indication of the second time slot indicates unavailability for the procedure.

8. The method of claim 1, further comprising the steps of:
displaying a plurality of providers based on a profile of the patient; and
receiving an input indicative of the preferred provider among the plurality of providers.

9. The method of claim 8, wherein the profile comprises insurance information associated with the patient.

10. The method of claim 8 further comprising:
receiving information for the plurality of providers, the information for the plurality of providers comprising service area indications corresponding to the plurality of providers.

11. The method of claim 1, wherein the scheduling request comprises a procedure indication of the procedure,
wherein the production yield and the procedure time are determined based on the procedure indication, and
wherein the procedure information further comprises the procedure indication of the procedure.

12. A system for using positional information for a mobile vehicle to generate dynamic scheduling, comprising:
a memory; and
a processor coupled with the memory, the processor configured to:
receive a scheduling request for a procedure of a patient, the procedure configured to occur inside of, in proximity to, or in connection with the mobile vehicle;
obtain scheduling information for the mobile vehicle corresponding to a preferred provider;
dynamically receive the positional information from the mobile vehicle, the positional information comprising a current location of the mobile vehicle;
determine a first production per drive score for a first time slot of a plurality of time slots for the procedure based on a production yield for the procedure, a first estimated travel time to the procedure, a procedure time for the procedure, and the positional information;
determine a first availability level indication of the first time slot for the procedure based on the first production per drive score of the first time slot for the procedure and the scheduling information;
dynamically display the first availability level indication on a graphical user interface;
dynamically transmit, to the mobile vehicle, procedure information comprising the first time slot; and
display the procedure information on the graphical user interface.

13. The system of claim 12, wherein the scheduling request comprises a service location of the procedure,
wherein the first estimated travel time is determined based on a distance from the current location of the positional information to the service location, and
wherein the procedure information further comprises the service location.

14. The system of claim 12, wherein the first production per drive score is determined by:
$$the first production per drive score = \frac{the production yield for the appointment}{( the first estimated travel time + the procedure time )} .$$

15. The system of claim 14, wherein to determine the first availability level indication of the first time slot, the processor is configured to:
calculate an availability score by the first production per drive score divided by a production per unit time score; and
determine the first availability level indication by comparing the availability score with one or more thresholds.

16. The system of claim 12, wherein the first availability level indication is displayed using a color corresponding to the first availability level indication.

17. The system of claim 12, wherein the processor is further configured to:
dynamically determine a second production per drive score for a second time slot of the plurality of time slots for the procedure based on the production yield for the procedure, a second estimated travel time to the procedure, and the procedure time for the procedure;
dynamically determine a second availability level indication of the second time slot for the procedure based on the second production per drive score of the second time slot for the procedure; and
dynamically display the second availability level indication on a graphical user interface,
wherein the scheduling request comprises a service location of the procedure, and
wherein the second estimated travel time is determined based on a previous location of a previous procedure and the service location of the procedure.

18. The system of claim 17, wherein the second availability level indication of the second time slot for the procedure is further determined based on the second estimated travel time,
wherein in response to the second time slot for the procedure being within the second estimated travel time after a previous time slot for the previous procedure or a dynamic travel time to travel from the current location of the positional information to the service location, the second availability level indication of the second time slot indicates unavailability for the procedure.

19. The system of claim 12, wherein the processor is further configured to:
display a plurality of providers based on a profile of the patient; and
receive an input indicative of the preferred provider among the plurality of providers.

20. The system of claim 19, wherein the profile comprises insurance information associated with the patient.

21. The system of claim 19, wherein the processor is further configured to:
receive information for the plurality of providers, the information for the plurality of providers comprising service area indications corresponding to the plurality of providers.

22. The system of claim 12, wherein the scheduling request comprises a procedure indication of the procedure,
wherein the production yield and the procedure time are determined based on the procedure indication, and
wherein the procedure information further comprises the procedure indication of the procedure.
